SEQUENCE LISTING (1 sequence)

SEQ ID NO: 1 - protein, 501 aa, Homo sapiens
MVRKPVVSTISKGGYLQGNVNGRLPSLGNKEPPGQEKVQLKRKVTLLRGVSIIIGTIIGAGIFISPKGVLQNTGSVGMSLTIWTVCGVLSLFGALSYAELGTTIKKSGGHYTYILEVFGPLPAFVRVWVELLIIRPAATAVISLAFGRYILEPFFIQCEIPELAIKLITAVGITVVMVLNSMSVSWSARIQIFLTFCKLTAILIIIVPGVMQLIKGQTQNFKDAFSGRDSSITRLPLAFYYGMYAYAGWFYLNFVTEEVENPEKTIPLAICISMAIVTIGYVLTNVAYFTTINAEELLLSNAVAVTFSERLLGNFSLAVPIFVALSCFGSMNGGVFAVSRLFYVASREGHLPEILSMIHVRKHTPLPAVIVLHPLTMIMLFSGDLDSLLNFLSFARWLFIGLAVAGLIYLRYKCPDMHRPFKVPLFIPALFSFTCLFMVALSLYSDPFSTGIGFVITLTGVPAYYLFIIWDKKPRWFRIMSEKITRTLQIILEVVPEEDKL

(19) United States
(12) Patent Application Publication
(10) Pub. No.: US 2024/0425857 A1
(43) Pub. Date: Dec. 26, 2024

(54) Means and Methods for Chronic Wound Healing

(71) Applicants: VIB VZW, Gent (BE); Universiteit Gent, Gent (BE)

(72) Inventors: Kodimangalam Ravichandran, Gent (BE); Sophia Maschalidi, Gent (BE)

(21) Appl. No.: 18/705,718

(22) PCT Filed: Oct. 26, 2022
(86) PCT No.: PCT/EP2022/079987

§ 371 Date: Apr. 29, 2024

Related U.S. Application Data
(60) Provisional application No. 63/273,504, filed on Oct. 29, 2021.

(51) Int. Cl. C12N 15/113; A61K 31/7088; A61K 38/18; A61K 38/46; A61P 17/02; C07K 16/18

(52) U.S. Cl. CPC C12N 15/113; A61K 31/7088; A61K 38/1841; A61K 38/465; A61P 17/02; C07K 16/18; C12N 2310/11; C12N 2310/14; C12N 2310/3231; C12N 2310/3233; C12N 2310/531

(57) ABSTRACT

The present invention relates to the field of tissue regeneration, particularly the field of skin lesions such as wounds, more particularly chronic wounds. The invention provides wound healing compositions. Specifically, the invention provides molecules targeting the Slc7a11 transporter such as antibodies, shRNA, siRNA, locked nucleic acids, peptide nucleic acids and morpholinos for use to treat chronic wounds such as for example diabetic wounds.

### CROSS REFERENCE TO RELATED APPLICATIONS

This application is a national phase entry under 35 U.S.C. § 371 of International Patent Application PCT/EP2022/079987, filed Oct. 26, 2022, designating the United States of America and published in English as International Patent Publication WO2023/073046 on May 4, 2023, which claims the benefit under Article 8 of the Patent Cooperation Treaty to U.S. Provisional Patent Application Ser. No. 63/273,504, filed Oct. 29, 2021, the entireties of which are hereby incorporated by reference.

### INCORPORATION BY REFERENCE

The ST.26 XML Sequence listing named “10425-US-SequenceListing”, created on Oct. 26, 2022, and having a size of 4,096 bytes, is hereby incorporated herein by this reference in its entirety.

### FIELD OF THE INVENTION

The present invention relates to the field of tissue regeneration, particularly the field of skin lesions such as wounds, more particularly chronic wounds. The invention provides wound healing compositions. Specifically, the invention provides molecules targeting the Slc7a11 transporter for use to treat chronic wound healing such as for example diabetic wound healing.

### INTRODUCTION TO THE INVENTION

Billions of cells in the body are turned over via apoptosis on a daily basis. These dying cells are then recognized and removed by phagocytes via the process of ‘efferocytosis’¹. From a physiological standpoint, failures in efferocytosis have been associated with non-resolving inflammation leading to chronic inflammatory conditions^{2,3,4,5,6}. Dendritic cells are a heterogenous group of phagocytes present in nearly all tissues. Dendritic cells display a battery of phagocytic and pathogen recognition receptors and help maintain tissue homeostasis through the regulation of innate and adaptive immunity⁷. While apoptotic cell uptake by dendritic cells has long been recognized, this has primarily been studied in the context of the antigen presentation and generation of adaptive immunity^{8,9,10,11,12}. Compared to macrophages, much less is known about the molecular regulation of efferocytosis in dendritic cells and the contribution of dendritic cell efferocytosis to limiting inflammation.

As the body's largest organ, our skin acts as a barrier to protect internal tissues from extreme temperature, water loss, ultraviolet radiation, microbial and chemical insults, and injury. Tissue repair after skin injury involves clearance of apoptotic cells by phagocytes at the wound site, in turn, helping to resolve the inflammation and restore the barrier^{13,14}. Chronic non-healing wounds, such as those associated with diabetes, aging, or vascular disease severely affect the quality of life and pose a significant risk for infection¹⁵. Homeostasis in healthy skin is maintained by immune cells including dendritic cells, macrophages, and T cells populating the tissue. Langerhans cells (LCs) residing in the epidermis, together with dendritic cells in the dermis are in a state of surveillance for capturing dead cells or pathogens and presenting them to effector T cells during homeostasis^{16,17}. After skin injury, resident or recruited macrophages^{18,19} and neutrophils²⁰ have been positively and negatively linked to wound healing dynamics; however, the knowledge of dendritic cell contribution to injury repair is limited.

A chronic wound is one that fails to progress through a normal, orderly, and timely sequence of repair, or in which the repair process fails to restore anatomic and functional integrity after three months. Approximately 5 million patients in the United States suffer from chronic wounds. With the increased longevity, obesity, and diabetes, the problem of chronic wounds has increased, resulting in significant morbidity, lost time from work, and enormous health-care expenses. According to the American Diabetes Association, 25% of people with diabetes will suffer from a wound problem during their lifetime, and approximately 82,000 limb amputations for nontraumatic wounds were performed in people with diabetes in 2002. The Agency for Health Care Policy and Research reports that wound care for pressure ulcers uses $200 billion a year for hospitalization, durable medical goods, nursing home care, physicians, and transportation. Surgical treatment of diabetic wounds remains difficult and often insufficient, leading to high morbidity among those patients. Some chronic wounds can take decades to heal, thus contributing to secondary conditions such as depression. The five-year mortality rate after developing a diabetic ulcer is approximately 40%. Despite recent advances in our understanding of wound healing, the molecular mechanisms underlying impaired wound healing are not completely understood, and currently available methods also have shown limited efficacy (Perez-Favila A. et al (2019)Medicina (Kaunas) 55:714).

### SUMMARY OF THE INVENTION

In the present invention, while trying to understand how gene programs in dendritic cells are modified as they engulf apoptotic cells, we serendipitously discovered the role of the plasma membrane protein Slc7a11 as a novel brake on dendritic cell-mediated efferocytosis and reveal its relevance to cutaneous wound healing. We show that the use of pharmacological inhibitors of Slc7a11, siRNA knockdown of Slc7a11, or gene deletion of Slc7a11 enhancing the apoptotic cell uptake by dendritic cells and leads to an increased wound closure in mammals.

In one aspect the invention provides a combination comprising an inhibitor of Slc7a11 and apoptotic cells.

In yet another aspect the invention provides a combination comprising an inhibitor of Slc7a11 and apoptotic cells for use as a medicament.

In yet another aspect the invention provides a composition comprising an inhibitor of Slc7a11 and apoptotic cells for use to treat wound healing.

In another aspect the invention provides an inhibitor of Slc7a11 for use to treat chronic wound healing in mammals.

In another aspect the invention provides an inhibitor for use to treat chronic wound healing wherein the inhibitor is selected from a small compound binding Slc7a11, an antibody binding Slc7a11, a gapmer binding the mRNA of Slc7a11, a shRNA binding the mRNA of Slc7a11, a synthetic siRNA binding the mRNA of Slc7a11, an anti-sense oligonucleotide binding the mRNA of slc7a11 selected from a locked nucleic acid (LNA), a peptide nucleic acid (PNA) or a morpholino, a CRISPR, a TALEN, or a Zinc-finger nuclease targeting the Slc7a11 gene.

In a specific aspect said chronic wound healing is diabetic wound healing, arterial or venous wound healing or a pressure wound.

In another aspect the invention provides a composition consisting of an inhibitor of Slc7a11 as defined herein before and GDF-15 for use as a medicament.

In another aspect the invention provides a composition consisting of an inhibitor of Slc7a11 as defined herein before and GDF-15 for use to treat chronic wound healing.

In another aspect the invention provides a pharmaceutical composition comprising an inhibitor as defined herein before for use to treat a chronic wound.

In another aspect the invention provides a pharmaceutical composition comprising an inhibitor as defined herein before for use to treat a chronic wound healing such as diabetic wound healing, arterial or venous wound healing or a pressure wound.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-11: Slc7a11 acts as a break on dendritic cell engulfment of apoptotic cells.

FIG. 1A, Schematic of phagocytosis assays by bone-marrow derived dendritic cells (BMDC) fed with apoptotic human Jurkat cells, live cells, or plain beads (2.0 μm) (left). RNAseq was performed on sorted engulfing BMDC after 4 hours of co-culture with targets. Histograms illustrate Yellow Green (beads) and CypHer5E (cells) fluorescence in efferocytic DCs.

FIGS. 1B and 1C, Genes associated with transporter activity that were modulated uniquely after efferocytosis in dendritic cells were categorized by functionality (b), and grouped by protein family (c). Data are from four independent experimental replicates per condition.

FIG. 1D, The SLC7 family gene expression was measured by qRT-PCR after phagocytosis of plain beads versus apoptotic cell uptake as in a. For clarity, Slc7a5 and Slc7a11 are highlighted in blue. Data represent 3 biological replicates per condition, ****P=0.0001, two-way analysis of variance (ANOVA) with Sidak's multiple comparison test. ND, not detected

FIG. 1E, Sl7a11 siRNA targeting increases efferocytosis of pHrodo Green-stained apoptotic cells as illustrated with live cell imaging analysis by Incucyte.

FIG. 1F, Treatment with erastin, a pharmacological inhibitor of Slc7a11, promotes apoptotic cell uptake by dendritic cells as shown over the time course.

FIG. 1G, Schematic of Slc7a11 WT and Slc7a11 KO mice and the phagocytes isolated from bone marrow or spleen.

FIG. 1H, Kinetics of efferocytosis by Slc7a11 WT and Slc7a11 KO BMDCs treated with erastin or vehicle. All live-cell imaging data are expressed as mean±SEM with n=4-8, ****p<0.00001; One-way ANOVA with Tukey's multiple comparisons test.

FIG. 11, Percentage of phagocytosis of TAMRA-or CypHer5E-labelled apoptotic Jurkat cells was compared between control and Slc7a11-null BMDCs (left panels). Percentage of phagocytosis of CypHer5E-labelled apoptotic Jurkat cells was compared between control and Slc7a11-null cDCs, which were previously isolated from the spleens of mice with the respective genotypes. Dendritic cells were incubated with apoptotic targets at a 1:5 phagocyte: target ratio for 4 hours. Data from n=3-5 biological replicates *p<0.05; **p<0.01; One-way ANOVA with Dunnett's multiple comparisons test.

All live-cell imaging data (e, f, h) are expressed as mean±SEM with n=4-8, ****p<0.00001; One-way ANOVA with Tukey's multiple comparisons test. ABCs, ATP-binding cassette transporters; GPCRs, G-protein-coupled receptors.

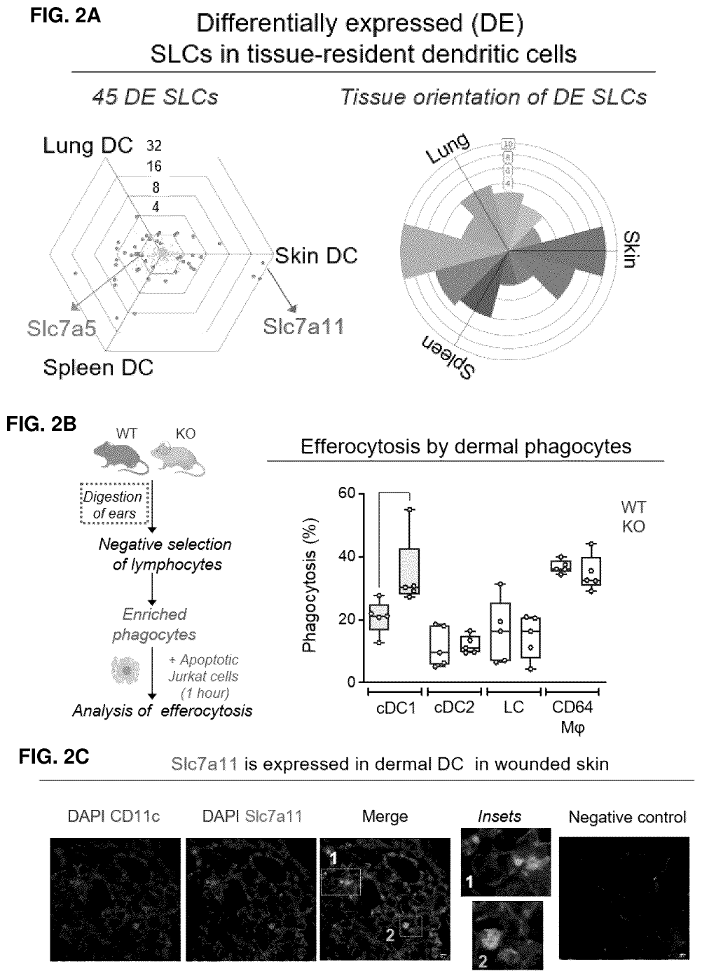

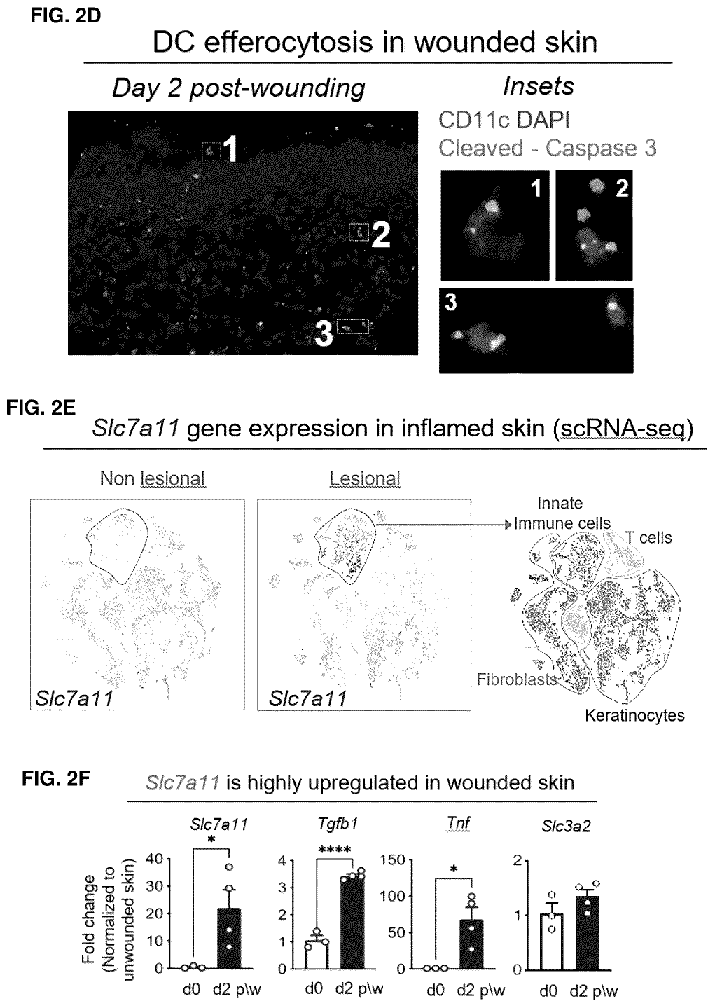

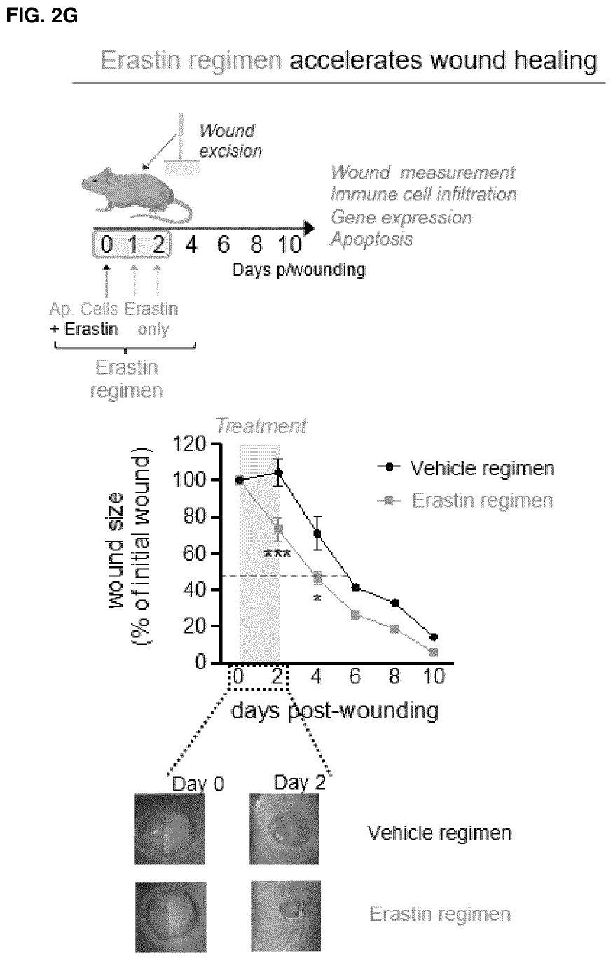

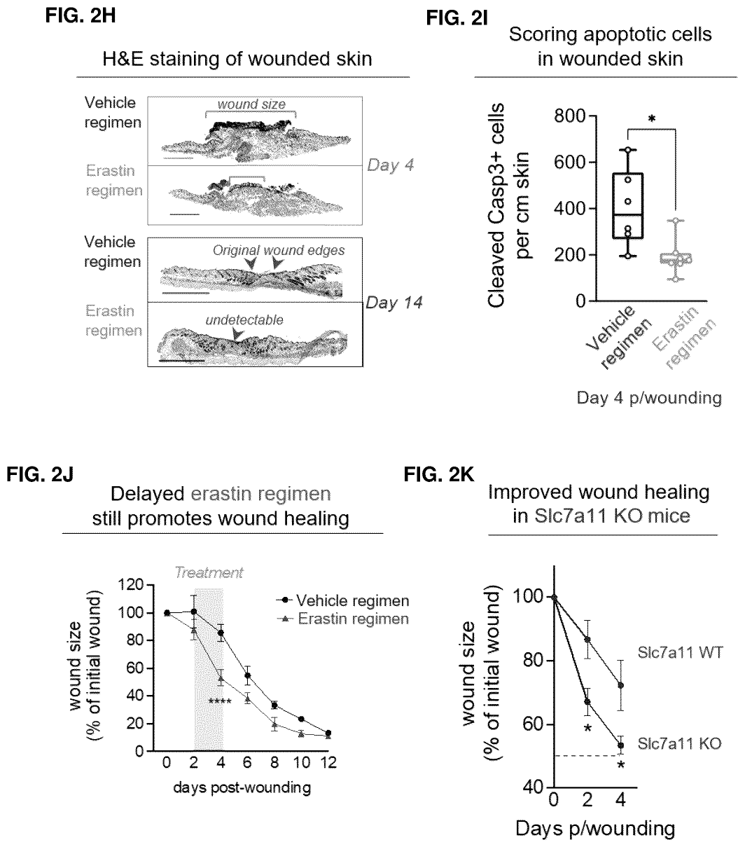

FIGS. 2A-2K: Accelerated skin wound healing in the context of Slc7a11 blockade

FIG. 2A, Hexagonal Tri-wise diagram (left) to visualize relative expression levels of differentially expressed SLCs (DE) between dendritic cells from lung, spleen and skin. Differentially expressed (DE) SLC genes (orange) and not differentially expressed (grey) are shown. The proximity represents upregulation in more than one population, and the distance from the origin represents the strength of this upregulation. Genes that are ≥32-fold expressed are plotted on the outer grid line. Rose plot (right) depicting the 45 DE Slc genes (hence all orange dots) grouped in bins and in the DC subtype in which they are most expressed.

FIG. 2B, Dermal phagocytes enriched from the ears of Slc7a11-deficient and control littermates, were incubated with apoptotic Jurkat cells and phagocytosis was evaluated in the respective cell types. (n=5 per group; *p<0.05, with unpaired t-test).

FIGS. 2C and 2D, Localization of CD11c⁺ phagocytes (red) and Slc7a11 (green) in skin sections from wounded (d2 post-wounding) skin (c). Dotted lines represent insets. Nuclei were stained with DAPI (4′, 6-diamidino-2-phenylindole; blue). Scale bar: 10 μm. CD11c⁺ phagocytes (red) capturing cleaved caspase-3 positive (green) corpses (d). Nuclei were stained with DAPI (4′, 6-diamidino-2-phenylindole; blue). Scale bar: 50 μm.

FIG. 2E, Annotated UMAP clustering of total live skin cells isolated from non-lesional (NL) and lesional (L) skin (see Methods) highlighting the expression of Slc7a11. Right panel depicts tSNE plot of cells as clustered in lesional skin after scRNAseq analysis.

FIG. 2F, mRNA from total skin lysates of unwounded and wounded mice analyzed by RT-qPCR for the indicated genes (n=3-4 per condition; *p<0.05; ****p<0.0001, with unpaired t-test).

FIG. 2G, Wound healing dynamics of wild-type mice treated with erastin or vehicle regimen consisting of a single administration to the wound site of apoptotic cells (Ap. cells) at day 0 along with erastin or DMSO vehicle given on day 0 to day 2. Data show one out of 4 independent experiments with n=8 mice per group) after full-thickness wounding with an 8 mm punch biopsy (*p<0.05; ***p<0.001 between groups; Two-way ANOVA with multiple comparisons).

FIGS. 2H and 2I, H&E-stained skin sections of wounds from mice on the erastin or vehicle regimen at different days post-wounding. Scale bars: 100 μm (h,) Quantification of apoptotic cleaved caspase-3⁺ cells (n=6-7 mice per condition; (*p<0.5 with unpaired t-test).

FIG. 2J, Wound healing kinetics comparing the efficacy of erastin regimen at different stages of wound repair (n=10 mice per group); ****p<0.0001; Two-way ANOVA with multiple comparisons.

FIG. 2K, Wound healing dynamics comparing Slc7a11 WT and Slc7a11 KO mice after full-thickness wounding. Mice received a single administration of apoptotic cells on time of wounding (n=6 per group, *p<0.05, between groups on the indicated days post-wounding; Two-way ANOVA with multiple comparisons).

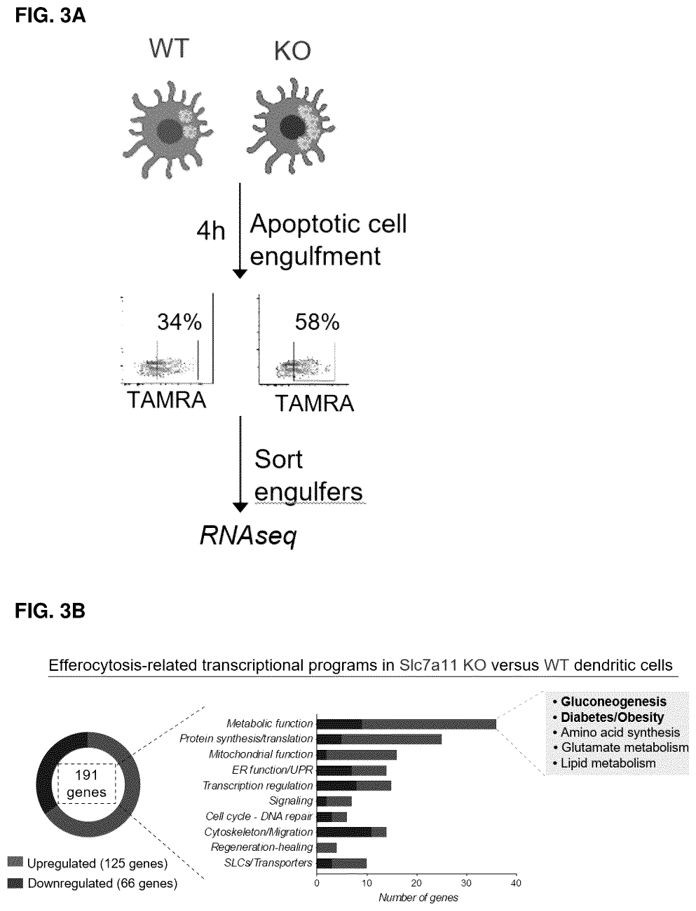

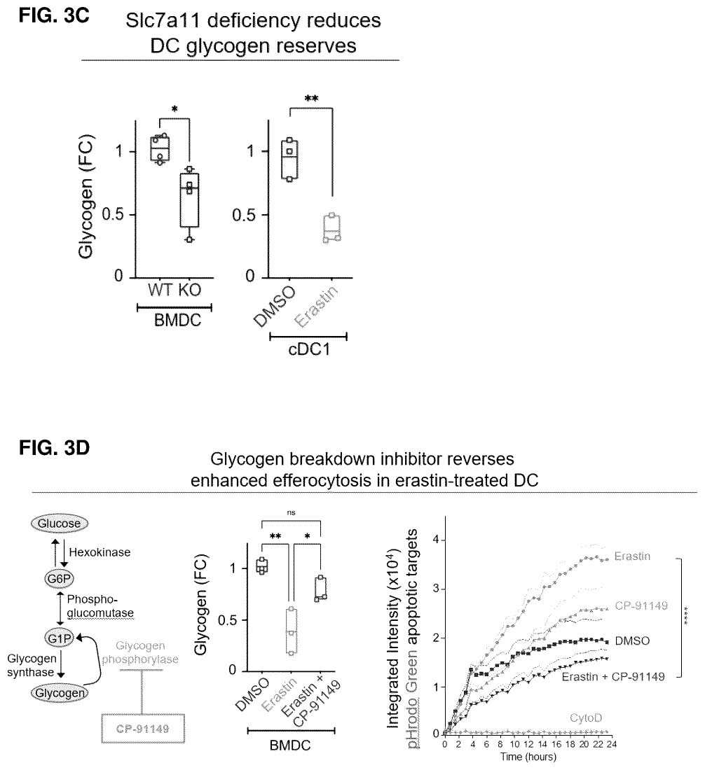

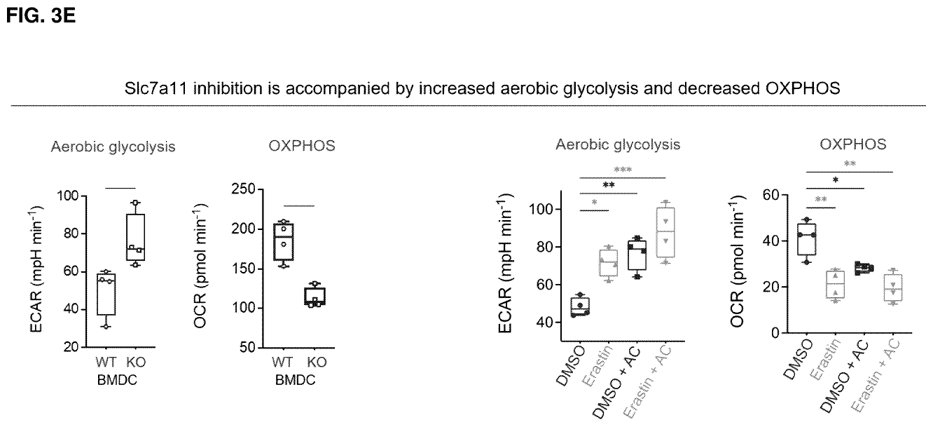

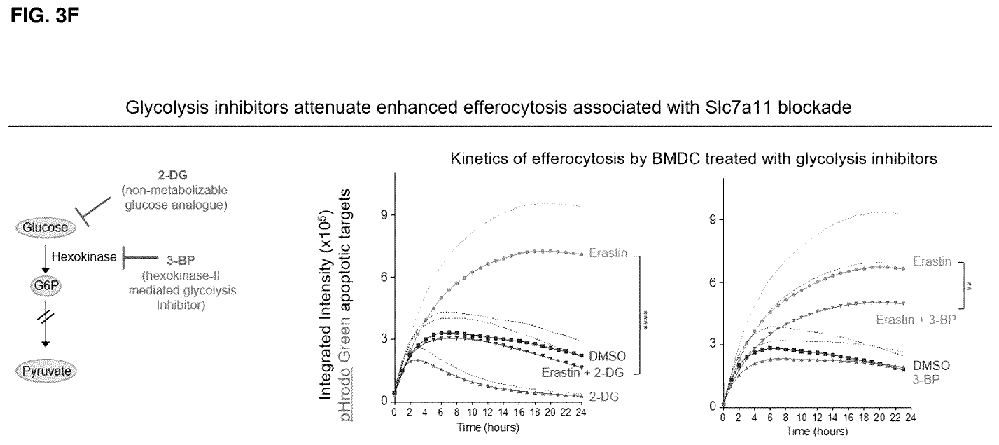

FIGS. 3A-3F: Glycolysis and glycogen reserves fuel enhanced efferocytosis

FIG. 3A, Schematic of the RNA-seq analysis of Slc7a11-deficient dendritic cells engulfing apoptotic cells. Slc7a11-deficient or control BMDC were cultured with TAMRA-labelled apoptotic Jurkat cells for 4 h. Phagocytes were collected and TAMRAt cells (i.e., engulfers) were sorted directly into lysis buffer for RNA-seq analysis. The contour plot images are representative of four independent experiments.

FIG. 3B, Pathway analysis of differentially expressed genes that are regulated in Slc7a11 KO engulfers compared to WT engulfers. Some genes falling under the transcriptional programs of gluconeogenesis and diabetes/obesity (depicted in bold and highlighted in grey square) were selected for further analysis. Data are from 3-4 independent experimental replicates.

FIG. 3C, Measurement of glycogen in lysates of dendritic cell populations from WT mice treated with erastin or from Slc7a11 KO mice. (*p<0.05; **p<0.01; via unpaired t-test or One-way ANOVA with Tukey's multiple comparisons test). Results are expressed as fold change (FC) from WT or DMSO-treated dendritic cells. ns, non-significant.

FIG. 3D, Schematic of glycogen metabolism pathway indicating the glycogenolysis step inhibited by CP-91149 (left). Impact on glycogen levels of BMDC treated with erastin or CP-91149 or both (middle panel). Kinetics of efferocytosis after erastin or erastin +CP-91149 treatment (right). DMSO was used as a vehicle control and CytoD as a negative control for efferocytosis. All live-cell imaging data are expressed as mean±SEM with n=3-5, (*p<0.05; ****p<0.00001. One-way ANOVA with Tukey's multiple comparisons test).

FIG. 3E, Increased aerobic glycolysis in dendritic cells during apoptotic cell clearance and Slc7a11 inhibition. Glycolysis and OXPHOS were measured at resting dendritic cells or during efferocytosis using Seahorse XF) via extracellular acidification rate (ECAR) and oxygen consumption rate (OCR). Data represent means±SEM of n=4; *p<0.05; **p<0.01; ***p<0.001; ****p<0.0001 via unpaired t-test or Two-way ANOVA with Tukey's multiple comparisons test).

FIG. 3F, Schematic of aerobic glycolysis pathway with respective inhibitors and kinetics of efferocytosis by BMDC treated the indicated glycolysis inhibitors. All live-cell imaging data are expressed as mean±SEM with n=4-5, (**p<0.01; ****p<0.00001. One-way ANOVA with Tukey's multiple comparisons test).

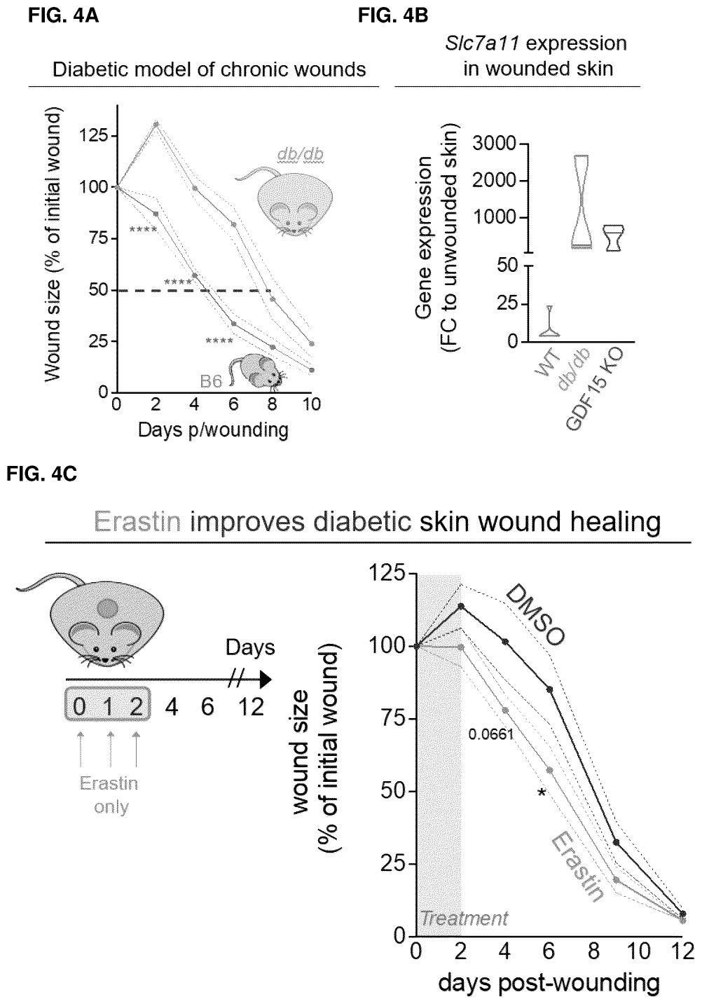

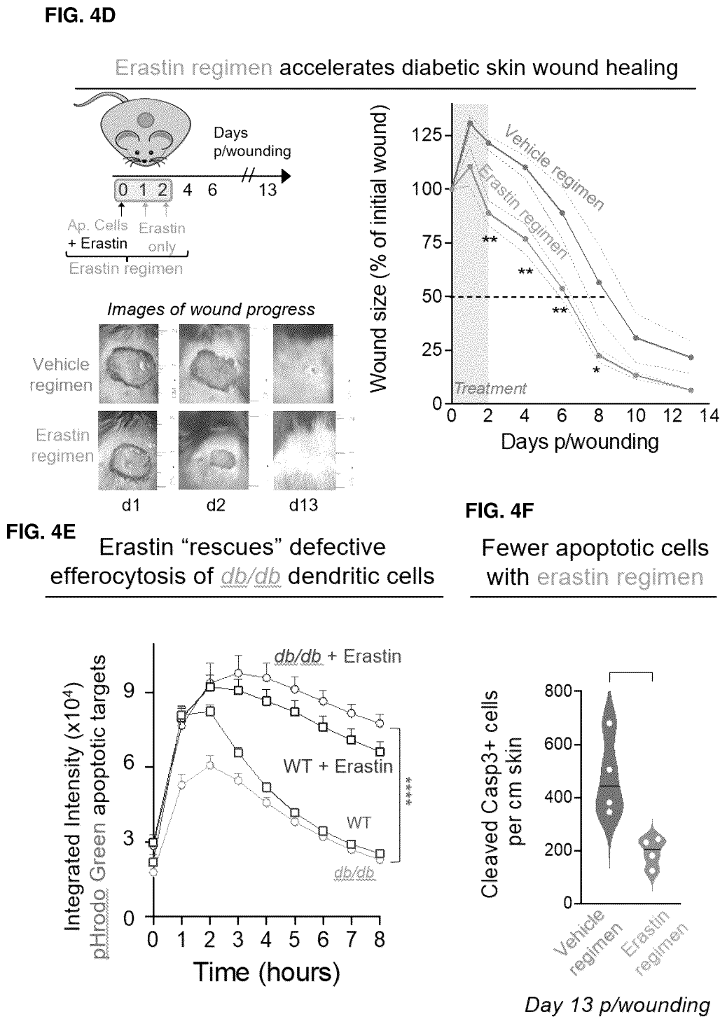

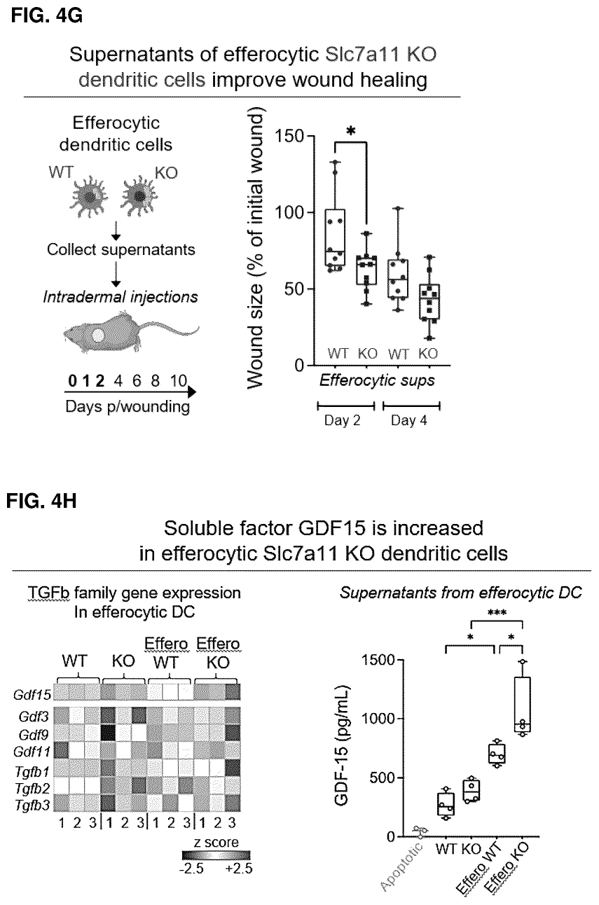

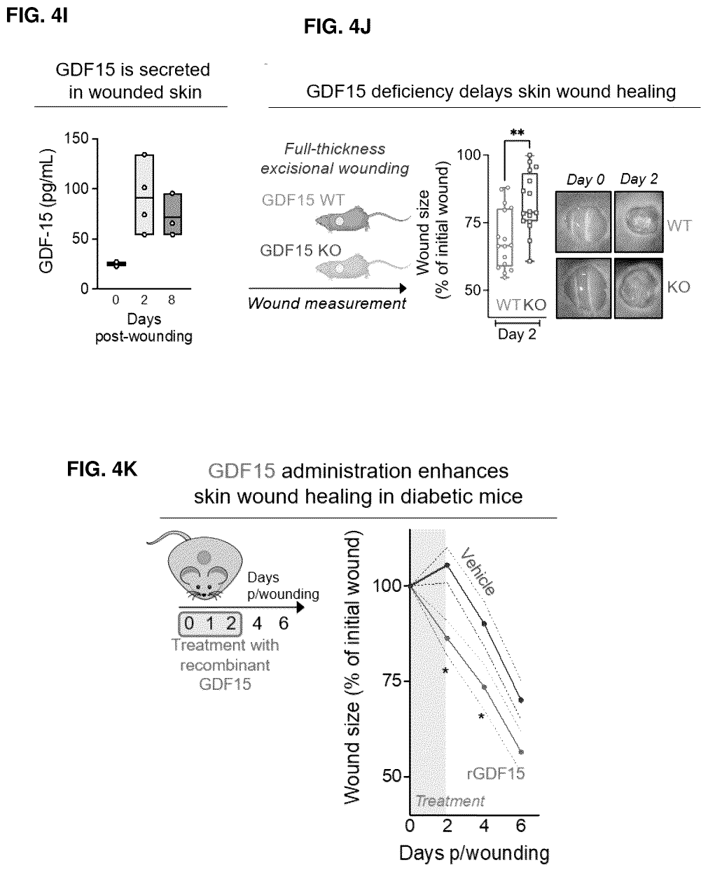

FIGS. 4A-4K: Slc7a11 inhibition and GDF15 promote diabetic skin wound healing

FIG. 4A, Delayed cutaneous wound healing in diabetic (db/db) mice versus normoglycemic (86) mice. (n=11 per group, ****p<0.0001, between groups on respective days post-wounding; Two-way ANOVA with multiple comparisons).

FIG. 4B, Total skin lysates were prepared from unwounded and wounded (d4 post-wounding) in WT, db/db and GDF15 KO mice, mRNA was isolated, followed by RT-qPCR analysis for Slc7a11. (n=4-6 per group; data are presented as fold change to Slc7a11 expression in unwounded skin per group).

FIG. 4C, Erastin alone improves skin wound healing in diabetic mice. Wound healing dynamics of db/db mice treated water soluble Imidazole Ketone Erastin (Erastin) or DMSO for vehicle control (n=7-8 mice per group *p<0.05 between groups on respective days post-wounding; Two-way ANOVA with multiple comparisons).

FIG. 4D, Erastin regimen accelerates skin wound healing in diabetic mice. Wound healing dynamics of db/db mice treated with erastin regimen (or vehicle). Data shown represent one of 3 independent experiments with n=7-8 mice per group after full-thickness wounding with an 8 mm punch biopsy (*p<0.05 or **p<0.01 between groups on the indicated days post-wounding; Two-way ANOVA with multiple comparisons). (Ap. Cells refers to Apoptotic cells)

FIG. 4E, Slc7a11 inhibition via erastin enhances efferocytosis in dendritic cells from diabetic mice. Efferocytosis data are expressed as mean±SEM with n=4-7, ***p<0.001 ****p<0.0001. One-way ANOVA with Tukey's multiple comparisons test.

FIG. 4F, Quantification of the number of apoptosis cells within wound biopsy that stain positive for cleaved caspase-3 in the wounded skin of db/db mice treated with erastin or vehicle (n=4 mice per condition; (* p<0.5 with unpaired t-test).

FIG. 4G, Wound healing dynamics of wild-type mice (n=10 per group) treated with sups from efferocytic

Slc7a11 KO and control dendritic cells after full-thickness wounding (*p<0.05, between groups on indicated days post-wounding; Two-way ANOVA with multiple comparisons). All wound sizes are expressed as percentage of initial wound size. Data represent means±SEM.

FIG. 4H, (left) Heatmap showing modulation of genes belonging to TGFE superfamily. Data are from three independent experimental replicates per conditions and are scaled by row resulting in a z-score that centers around zero. (right) GDF-15 secretion was assessed by ELISA in the supernatants of WT and Slc7a11-KO efferocytic dendritic cells 12 hr after incubation with apoptotic targets, or apoptotic targets alone. n=4, *p<0.05 ***p<0.001. One-way ANOVA with Tukey's multiple comparisons test.

FIG. 4I, GDF15 levels in unwounded and wounded skin lysate (d2 and d8 post-wounding) by ELISA,

FIG. 4J, Wound healing dynamics comparing GDF15 KO and littermate control mice after full-thickness wounding (n=16 per group, **p<0.01, between two groups via unpaired t-test).

FIG. 4K, Topical administration of recombinant GDF15 in the wounded skin of diabetic mice boosts healing. (n=10-11 per group, *p<0.05, between groups on the indicated days post-wounding; Two-way ANOVA with multiple comparisons).

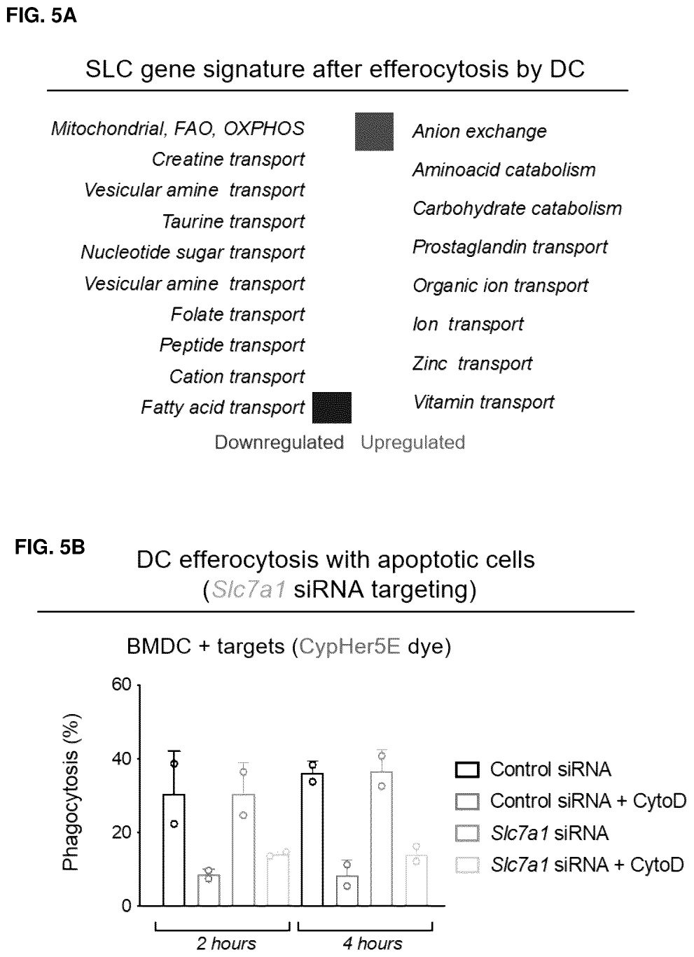

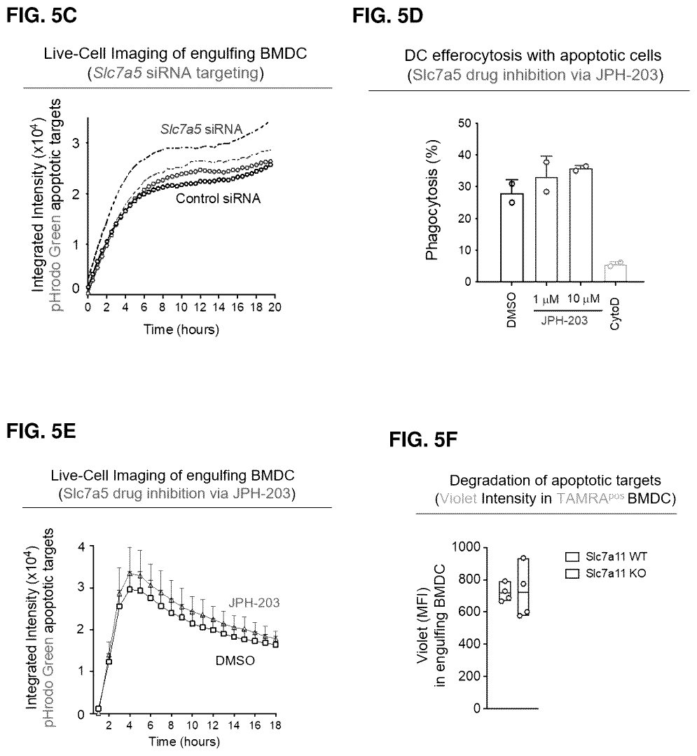

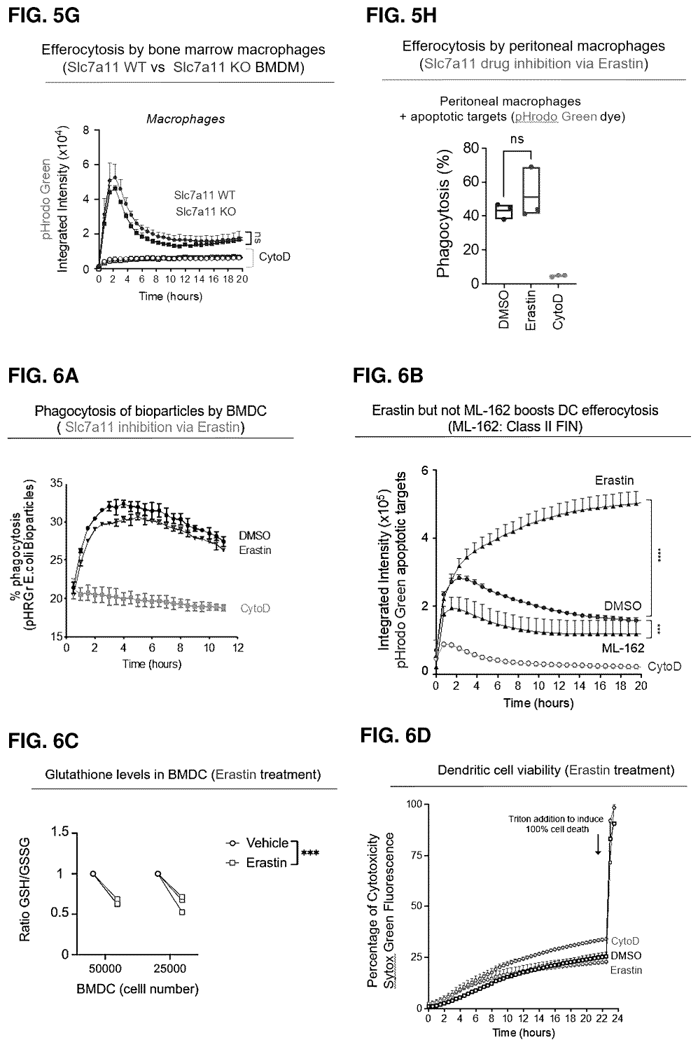

FIGS. 5A-5H: Analyzing amino acid transporters during dendritic cell efferocytosis. FIG. 5A, SLC programs modulated in dendritic cells during efferocytosis of apoptotic cells compared to sterile phagocytosis. RNAseq was performed on primary BMDCs after engulfment apoptotic human Jurkat cells or beads. The heatmap illustrates SLCs upregulated and downregulated during dendritic cell efferocytosis.

FIG. 5B, Uptake of CypHerSE-labelled apoptotic Jurkat cells by dendritic cells (BMDC) silenced for Slc7a1 expression.

FIG. 5C, Slc7a5 siRNA targeting in dendritic cells does not significantly affect apoptotic cell uptake as assessed by Incucyte Live-cell imaging. Data are expressed as mean±SD with n=4.

FIG. 5D, Efferocytosis of TAMRA-labelled apoptotic Jurkat cells by dendritic cells treated with different concentrations of the Slc7a5 inhibitor, JPH-203. (n=2 per condition).

FIG. 5E, Kinetics of efferocytosis by BMDC treated with 3 μM JPH-203. Live-cell imaging data are expressed as mean±SEM with n=3.

FIG. 5F, Measurement of degradation of TAMRA (pH insensitive) and Cell Trace Violet (pH sensitive) co-labelled apoptotic targets after efferocytosis by Slc7a11 KO and WT dendritic cells. Dendritic cells were incubated with apoptotic targets at a 1:5 phagocyte: target ratio for four hours.

FIG. 5G, Kinetics of efferocytosis by Slc7a11 WT and Slc7a11 KO bone marrow-derived macrophages with or without erastin treatment. All live-cell imaging data are expressed as mean±SEM with n=4, ns: not significant. One-way ANOVA with Tukey's multiple comparisons test.

FIG. 5H, Efferocytosis by peritoneal macrophages after Slc7a11 inhibition via erastin. (n=3 per condition; ns: not significant, with unpaired t-test).

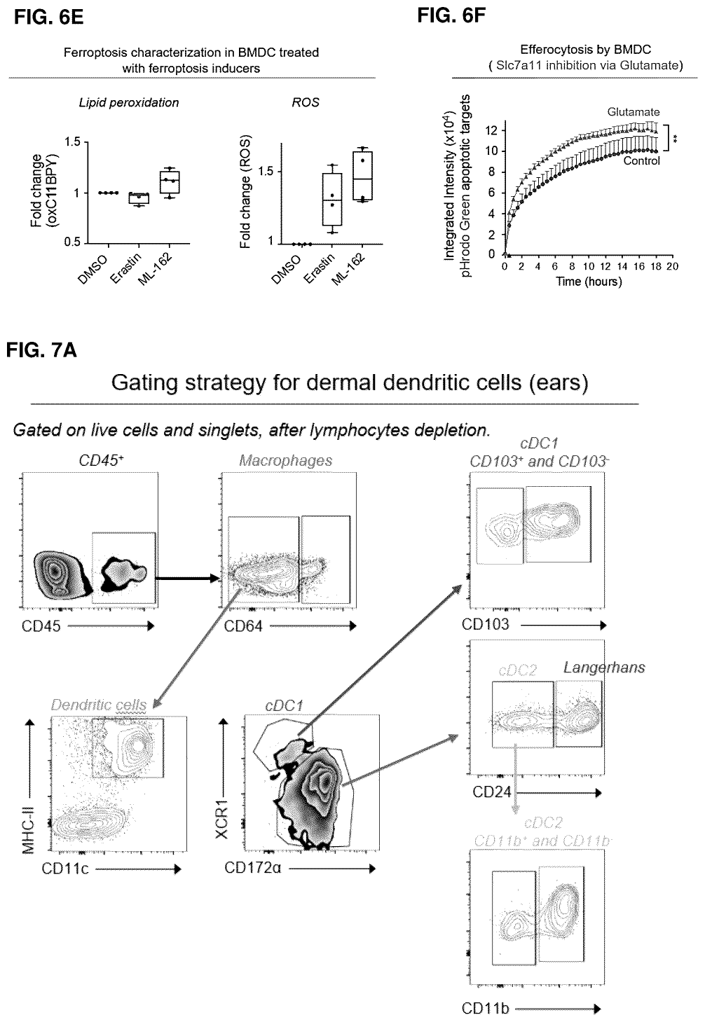

FIGS. 6A-6F: Ferroptosis inducer and DC efferocytosis

FIG. 6A, Phagocytosis of E. coli bioparticles by dendritic cells measured at different time points with or without erastin treatment.

FIG. 6B, Ferroptosis inducer ML-162 does not enhance efferocytosis by dendritic cells. Live-cell imaging data are expressed as mean±SEM with n=4-6, ***p<0.001; ****p<0.0001. One-way ANOVA with Tukey's multiple comparisons test).

FIG. 6C, Measurement of glutathione levels in dendritic cells treated with erastin.

FIG. 6D, Assessment of erastin drug cytotoxicity in dendritic cells by measuring Sytox Green fluorescence.

FIG. 6E, Measurement of lipid peroxidation and ROS in dendritic cells treated with ferroptosis inducers.

FIG. 6F, Kinetics of efferocytosis by dendritic cells treated with glutamate. Live-cell imaging data are expressed as mean±SEM with n=4-6, **p<0.01. One-way ANOVA with Tukey's multiple comparisons test).

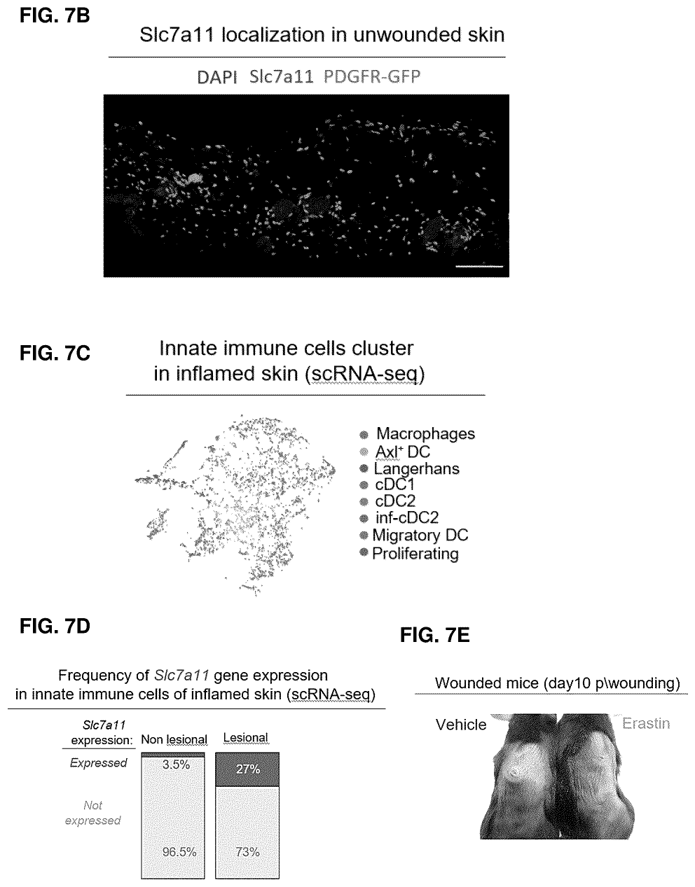

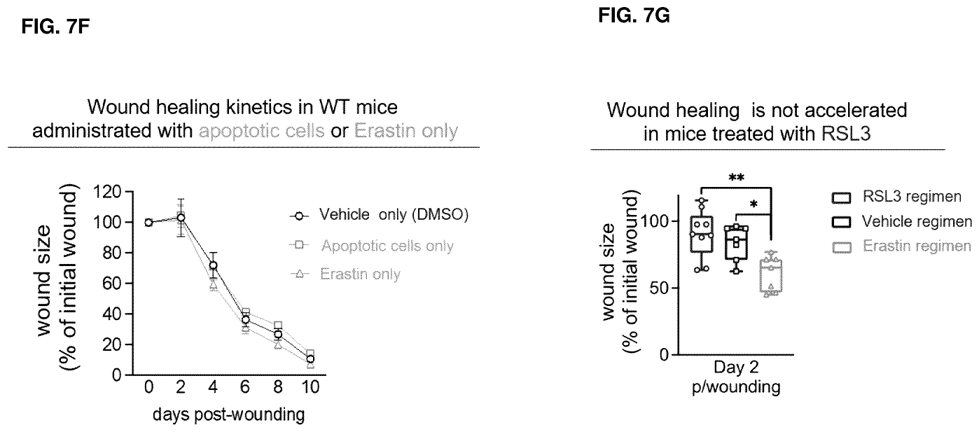

FIGS. 7A-7G: Analysis of dermal DCs and wound healing

FIG. 7A, Gating strategy of enriched phagocytes after digestion of ears and depletion of lymphocytes.

FIG. 7B, Immunofluorescent images of skin sections from unwounded PDGFR-GFP mice depicting Slc7a11-positive (red) cells. Nuclei were stained with DAPI (4′, 6-diamidino-2-phenylindole; blue). Scale bar: 50 μm.

FIG. 7C, Annotations of innate immune cell populations arising in lesional skin.

FIG. 7D, Frequencies of Slc711 expression in innate immune cells of lesional and non-lesional skin.

FIG. 7E, Representative images of wounds of mice treated with erastin or vehicle at day 10 post-wounding.

FIG. 7F, Wound healing dynamics comparing WT (C57B/6) mice after full-thickness wounding with an 8-mm punch biopsy and a single topical administration of apoptotic targets at the day of wounding (n=8 per group) or erastin only at day 0 till day 2 (n=8 per group).

FIG. 7G, Comparison of wound closure at day 2 post wounding in WT mice treated with RSL3 regimen versus erastin regimen. All mice received a single topical administration of apoptotic targets at the day of wounding (n=7-9 per group, *p<0.05; **p<0.01. One-way ANOVA with Tukey's multiple comparisons test).

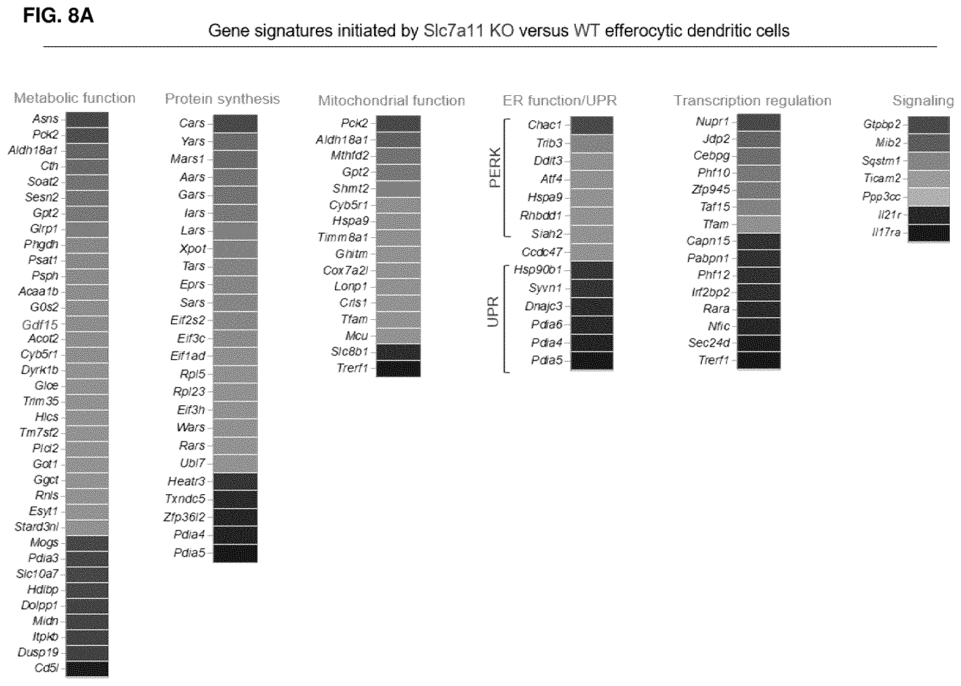

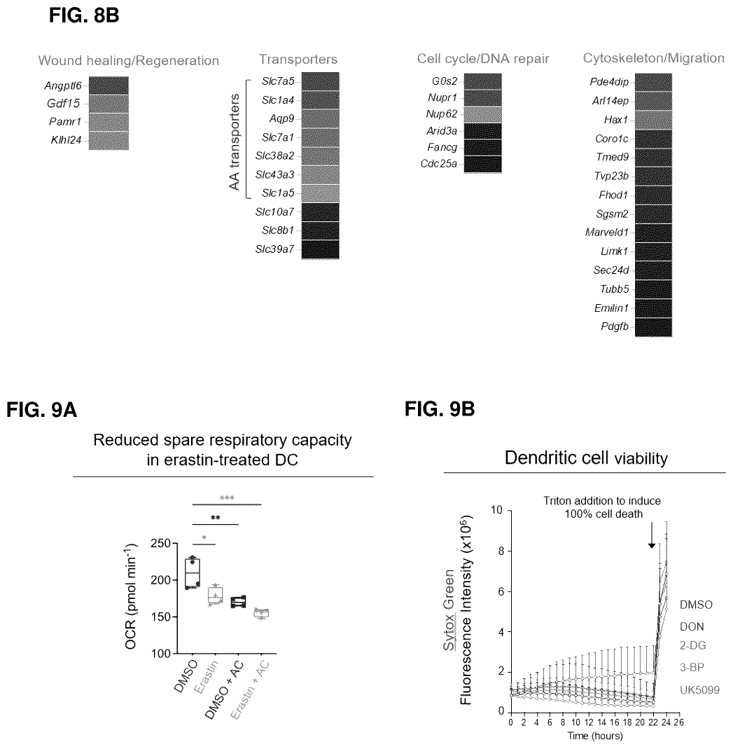

FIGS. 8A and 8B: Gene expression patterns in Slc7a11 KO efferocytic dendritic cells

FIGS. 8A and 8B, Heat maps comparing efferocytic Slc7a11 KO versus WT dendritic cells showing upregulation and downregulation of genes associated with metabolic and mitochondrial function, protein synthesis, ER homeostasis, transcription regulation, signaling, wound healing, cell cycle, migration and other transporters. Data are from 3-4 independent experimental replicates. Gdf15 falls under the transcriptional programs of metabolic function and regeneration, and is highlighted in red.

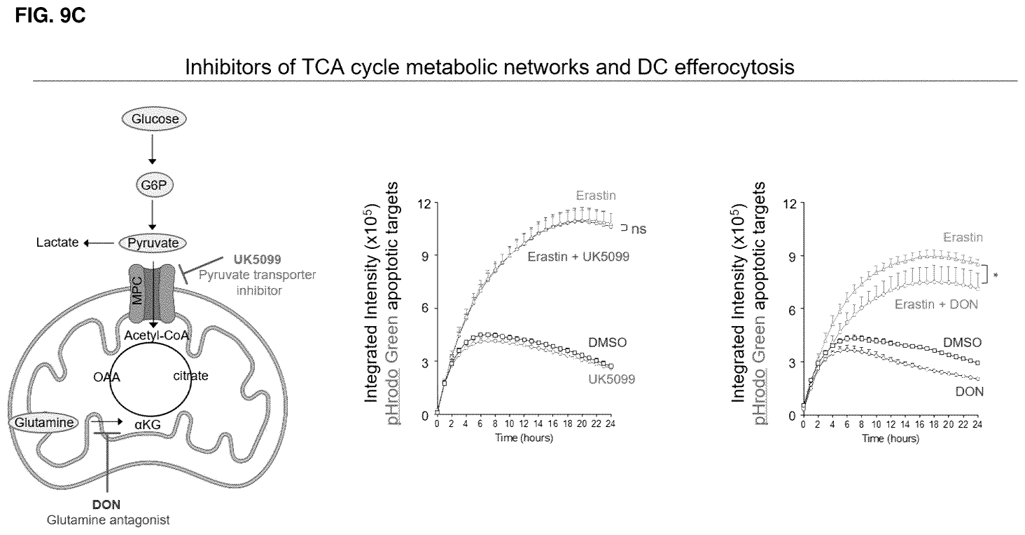

FIGS. 9A-9C: Metabolic inhibitors and DC efferocytosis

FIG. 9A, Spare capacity was measured in dendritic cells after vehicle or erastin-treatment or during efferocytosis (with Seahorse XF) using oxygen consumption rate (OCR). Data from n=4; *p<0.05; ** p<0.01; ***p<0.001, via One-way ANOVA with Tukey's multiple comparisons test).

FIG. 9B, Assessment of cytotoxicity of drugs tested on dendritic cells by measuring Sytox Green fluorescence.

FIG. 9C, Kinetics of efferocytosis by WT BMDC treated with the indicated inhibitors DON or UK5099. All live-cell imaging data are expressed as mean±SEM with n=3, (*p<0.05; ns: not significant via One-way ANOVA with Tukey's multiple comparisons test).

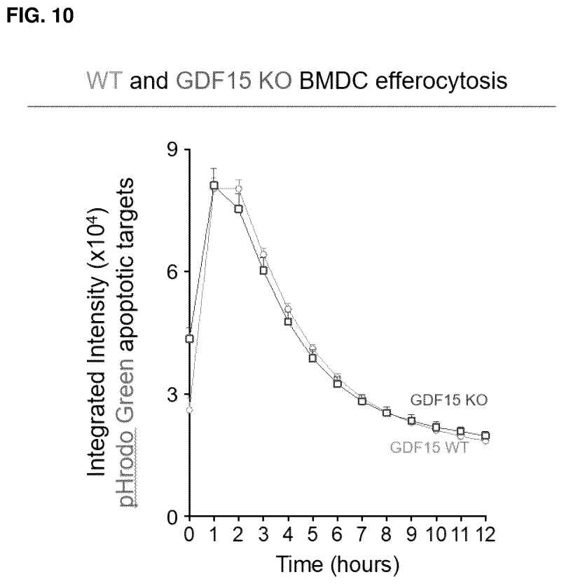

FIG. 10: GDF15 deficiency does not impair DC efferocytosis

FIG. 10, Kinetics of efferocytosis by GDF15 KO and WT BMDC with or without erastin treatment. All live-cell imaging data are expressed as mean±SEM with n=6-9, One-way ANOVA with Tukey's multiple comparisons test).

### DETAILED DESCRIPTION OF THE INVENTION

The present invention will be described with respect to particular embodiments and with reference to certain drawings but the invention is not limited thereto but only by the claims. Any reference signs in the claims shall not be construed as limiting the scope. Of course, it is to be understood that not necessarily all aspects or advantages may be achieved in accordance with any particular embodiment of the invention. Thus, for example those skilled in the art will recognize that the invention may be embodied or carried out in a manner that achieves or optimizes one advantage or group of advantages as taught herein without necessarily achieving other aspects or advantages as may be taught or suggested herein. The invention together with features and advantages thereof, may best be understood by reference to the following detailed description when read in conjunction with the accompanying drawings. The aspects and advantages of the invention will be apparent from and elucidated with reference to the embodiment(s) described hereinafter. Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may.

Similarly, it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim.

Where an indefinite or definite article is used when referring to a singular noun e.g. “a” or “an”, “the”, this includes a plural of that noun unless something else is specifically stated. Where the term “comprising” is used in the present description and claims, it does not exclude other elements or steps. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments, of the invention described herein are capable of operation in other sequences than described or illustrated herein. The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 4^{th} ed., Cold Spring Harbor Press, Plainsview, New York (2012); and Ausubel et al., Current Protocols in Molecular Biology (Supplement 114), John Wiley & Sons, New York (2016), for definitions and terms of the art. Unless defined otherwise, all technical and scientific terms used herein have the same meaning as commonly understood by one of ordinary skill in the art (e.g. in molecular biology, biochemistry, structural biology, and/or computational biology).

The terms “protein”, “polypeptide”, and “peptide” are interchangeably used further herein to refer to a polymer of amino acid residues and to variants and synthetic analogues of the same. A “peptide” may also be referred to as a partial amino acid sequence derived from its original protein, for instance after tryptic digestion. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers. This term also includes posttranslational modifications of the polypeptide, such as glycosylation, phosphorylation and acetylation. Based on the amino acid sequence and the modifications, the atomic or molecular mass or weight of a polypeptide is expressed in (kilo) dalton (kDa). A “protein domain” is a distinct functional and/or structural unit in a protein. Usually a protein domain is responsible for a particular function or interaction, contributing to the overall role of a protein. Domains may exist in a variety of biological contexts, where similar domains can be found in proteins with different functions.

By “isolated” or “purified” is meant material that is substantially or essentially free from components that normally accompany it in its native state.

“Homologue”, “Homologues” of a protein encompass peptides, oligopeptides, polypeptides, proteins and enzymes having amino acid substitutions, deletions and/or insertions relative to the unmodified protein in question and having similar biological and functional activity as the unmodified protein from which they are derived. The term “amino acid identity” as used herein refers to the extent that sequences are identical on an amino acid-by-amino acid basis over a window of comparison. Thus, a “percentage of sequence identity” is calculated by comparing two optimally aligned sequences over the window of comparison, determining the number of positions at which the identical amino acid residue (e.g., Ala, Pro, Ser, Thr, Gly, Val, Leu, Ile, Phe, Tyr, Trp, Lys, Arg. His, Asp, Glu, Asn, Gln, Cys and Met, also indicated in one-letter code herein) occurs in both sequences to yield the number of matched positions, dividing the number of matched positions by the total number of positions in the window of comparison (i.e., the window size), and multiplying the result by 100 to yield the percentage of sequence identity. A “substitution”, or “mutation”, or “variant” as used herein, results from the replacement of one or more amino acids or nucleotides by different amino acids or nucleotides, respectively as compared to an amino acid sequence or nucleotide sequence of a parental protein or a fragment thereof. It is understood that a protein or a fragment thereof may have conservative amino acid substitutions which have substantially no effect on the protein's activity.

One of the major complications of diabetes, which impacts 1 in 10 people worldwide, is the development of chronic non-healing wounds. A factor that contributes to dysregulated tissue repair is the continued presence of dying cells in the wound perpetuating the inflammation. In the present invention we have identified the membrane transporter Slc7a11 as a novel molecular ‘brake’ on efferocytosis, the process by which dying cells are removed. In the invention we show that Slc7a11 inhibition can accelerate diabetic wound healing. Initially, via transcriptomics of dendritic cells engulfing apoptotic cells, we noted the upregulation of several members of the Slc7 gene family. In subsequent functional studies, the cystine/glutamate antiporter Slc7a11 emerged as a negative regulator of efferocytosis, with pharmacological inhibition, siRNA knockdown, or gene deletion of Slc7a11 enhancing the apoptotic cell uptake by dendritic cells. Interestingly, Slc7a11 was most highly expressed in skin dendritic cells, and scRNAseq of inflamed skin showed Slc711 upregulation in innate immune cells. In a mouse model of excisional skin wounding, we found that loss or silencing of Slc7a11 expression accelerated healing dynamics and reduced the apoptotic cell load in the wound. Mechanistic studies pointed to a link between Slc7a11, glucose homeostasis, and diabetes: first, Slc7a11-deficient dendritic cells relied on glycogen store-derived aerobic glycolysis for their improved efferocytosis; and second, transcriptomics of efferocytic Slc7a11-deficient dendritic cells identified genes linked to gluconeogenesis and diabetes.

When we tested the role of Slc7a11 in delayed wound healing associated with diabetic conditions, Slc7011 expression was upregulated in the skin of diabetic db/db mice, and Slc7a11 inhibition accelerated wound healing in the db/db mice. This faster healing was associated with the improved apoptotic cell removal in the wounds and release of the TGF-beta family member GDF15 from efferocytic dendritic cells. Collectively, our data identifies Slc7a11 as a negative regulator of efferocytosis and that removing this obstacle can improve wound healing, with significant implications for chronic wound healing such as diabetic wound healing.

The solute carrier family 7 member 11 (abbreviated as Slc7A11) gene encodes a member of a heteromeric, sodium-independent, anionic amino acid transport system that is highly specific for cysteine and glutamate. In this system, designated Xc(-), the anionic form of cysteine is transported in exchange for glutamate. Aliases for Slc7a11 are Solute carrier family 7 (anionic amino acid transporter light chain, Xc- system, member 11, calcium channel blocker resistance protein CCBR1, amino acid transport system Xc-, XCT and Cystine/Glutamate transporter.

For the avoidance of doubt the amino acid sequence of the human Slc7a11 protein is depicted in SEQ ID NO: 1

- amino acid sequence of the human Scl7a11 protein
SEQ ID NO: 1
NH2-mvrkpvvstiskggylqgnvngrlpslgnkeppgqekvqlkrkvtl
lrgvsiiigtiigagifispkgvlqntgsvgmsltiwtvcgvlslfgals
yaelgttikksgghytyilevfgplpafvrvwvelliirpaatavislaf
gryilepffiqceipelaiklitavgitvvmvlnsmsvswsariqifltf
ckltailiiivpgvmqlikgqtqnfkdafsgrdssitrlplafyygmyay
agwfylnfvteevenpektiplaicismaivtigyvltnvayfttinaee
lllsnavavtfserllgnfslavpifvalscfgsmnggvfavsrlfyvas
reghlpeilsmihvrkhtplpavivlhpltmimlfsgdldsllnflsfar
wlfiglavagliylrykcpdmhrpfkvplfipalfsftclfmvalslysd
pfstgigfvitltgvpayylfiiwdkkprwfrimsekitrtlqiilevvp
eedkl-COOH

Thus, in a first embodiment the invention provides the combination of an inhibitor of Slc7a11 and apoptotic cells.

In yet another embodiment the invention provides a combination of an inhibitor of Slc7a11 and apoptotic cells for use as a medicament.

In yet another embodiment the invention provides the combination of an inhibitor of Slc7a11 and apoptotic cells for use to treat wound healing.

In yet another embodiment the invention provides for an inhibitor of Slc7a11 for use to treat chronic wound healing in a mammal.

As used herein, the term “wound” includes an injury to any tissue, including, for example, acute, delayed, or difficult to heal wounds, and chronic wounds. Examples of wounds may include both open and closed wounds. Wounds include, for example, burns, incisions, excisions, lacerations, abrasions, puncture on penetrating wounds, surgical wounds, contusions, hematoma, crushing injuries, and ulcers. Also included are wounds that do not heal at expected rates, as is expected in diabetic patients. The term “wound” may also include for example, injuries to the skin and subcutaneous tissue initiated in different ways (e.g. pressure sores from extended bed rest and wounds induced by trauma) and with varying characteristics. Wounds may be classified into one of four grades depending on the depth of the wound: i) Grade I: wounds limited to the epithelium; ii) Grade II: wounds extending into the dermis; iii) Grade III: wounds extending into the subcutaneous tissue; and iv) Grade IV (or full-thickness wounds): wounds wherein bones are exposed (e.g. a bony pressure point such as the greater trochanter or the sacrum).

The term “partial thickness wound” refers to wounds that encompass Grades I-III; examples of partial thickness wounds include pressure sores, venous stasis ulcers, and diabetic ulcers. The present invention contemplates treating all wounds of a type that do not heal at expected rates, including, delayed-healing wounds, incompletely healing wounds, and chronic wounds.

By “wound that does not heal at an/the expected rate” is meant an injury to any tissue that does not heal in an expected or typical time frame, including delayed or difficult to heal wounds (including delayed or incompletely healing wounds), and chronic wounds. Examples of wounds that do not heal at the expected rate include ulcers such as diabetic ulcers, vasculitic ulcers, arterial ulcers, venous ulcers, venous stasis ulcers, burn ulcers, infectious ulcers, trauma-induced ulcers, pressure ulcers, decubitus ulcers, ulcerations associated with pyoderma gangrenosum, and mixed ulcers. Other wounds that do not heal at expected rates include dehiscent wounds.

As used herein, a delayed or difficult to heal wound may include, for example, a wound that is characterized at least in part by 1) a prolonged inflammatory phase, 2) a slow forming extracellular matrix, and/or 3) a decreased rate of epithelialization or closure.

The term “chronic wound” refers to a wound that has not healed. Wounds that do not heal within 6 weeks, for example, are considered chronic. Chronic wounds include, for example, pressure ulcers, decubitus ulcers, diabetic ulcers including diabetic foot and leg ulcers, slow or non-healing venous ulcers, venous stasis ulcers, arterial ulcers, vasculitic ulcers, burn ulcers, trauma-induced ulcers, infectious ulcers, mixed ulcers, and pyoderma gangrenosum. The chronic wound may be an arterial ulcer that comprises ulcerations resulting from complete or partial arterial blockage. The chronic wound may be a venous or venous stasis ulcer that comprises ulcerations resulting from a malfunction of the venous valve and the associated vascular disease.

As used herein, the term “dehiscent wound” refers to a wound, usually a surgical wound, which has ruptured or split open. In certain embodiments, a method of treating a wound that does not heal at the expected rate is provided wherein the wound is characterized by dehiscence.

In addition to the definition previously provided, the term “wound” may also include for example, injuries to the skin and subcutaneous tissue initiated in different ways (e.g., pressure sores from extended bed rest and wounds induced by trauma) and with varying characteristics.

In a specific embodiment the inhibitor for use to treat chronic wound healing in a mammal is selected from a small compound, an antibody, a gapmer, a shRNA, a synthetic siRNA, an anti-sense oligonucleotide selected from a locked nucleic acid (LNA), a peptide nucleic acid (PNA) or a morpholino, a CRISPR, a TALEN, or a Zinc-finger nuclease targeting slc7a11

In a further embodiment the chronic wound healing is diabetic wound healing, arterial or venous wound healing or a pressure wound.

The vast majority of chronic wounds fall into three main categories: venous and arterial ulcers, pressure ulcers and diabetic ulcers (see Zhao R. et al (2016) Int. J. Mol. Sci. 17, 2085).

In yet another embodiment the invention provides a pharmaceutical composition comprising an inhibitor of Slc7a11 for use to treat a chronic wound.

System Xc-can be inhibited by many small molecules known in the art. Small chemical molecules such as erastin, sulfasalazine and sorafenib can inhibit system Xc- function and induce ferroptosis. Other molecules which are described to inhibit Xc- (approved for several conditions) are riluzole, cystine, glutamic acid and acetylcysteine. Still other small molecules inhibiting system Xc-are substituted N-acetyl-L-cysteine derivatives described in the claims of EP3066089B1.

A cellular assay to monitor the effect of inhibitors of the Xc-system is for example the measurement of cystine uptake. Cystine uptake levels can be measured as a convenient assay for monitoring the inhibition of Slc7A11 activity. Cystine uptake can for example by analyzed following the sodium nitroprusside-based protocol previously described by Y. Gu et al (2017) Mol. Cell 67, 128-138.e7 (2017). Optionally the monitoring of cystine uptake is carried out in tandem by measuring the glutamate secretion which can be quantified using a variety of assays such as for example the Glutamate-Glo assay sold by Promega.

Mammals include humans, pets (e.g. dogs and cats), veterinary animals (e.g. horses, pigs, cows), livestock and research animals.

### DNA and RNA Inhibitors of the Slc7a11 Gene

In a specific embodiment it is an object of the invention to provide inhibitors of functional expression of the Slc7A11 target gene. Such inhibitors can act at the DNA level, at the RNA (i.e. gene product) level.

If inhibition is to be achieved at the DNA level, this may be done using gene therapy to knock-out or disrupt the Slc7A11 target gene. As used herein, a “knock-out” can be a gene knockdown or the gene can be knocked out by a mutation such as, a point mutation, an insertion, a deletion, a frameshift, or a missense mutation by techniques known in the art, including, but not limited to, retroviral gene transfer. Another way in which genes can be knocked out is by the use of zinc finger nucleases. Zinc-finger nucleases (ZFNs) are artificial restriction enzymes generated by fusing a zinc finger DNA-binding domain to a DNA-cleavage domain. Zinc finger domains can be engineered to target desired DNA sequences, which enable zinc-finger nucleases to target unique sequence within a complex genome. By taking advantage of endogenous DNA repair machinery, these reagents can be used to precisely alter the genomes of higher organisms. Other technologies for genome customization that can be used to knock out genes are meganucleases and TAL effector nucleases (TALENs, Cellectis bioresearch). A TALEN® is composed of a TALE DNA binding domain for sequence-specific recognition fused to the catalytic domain of an endonuclease that introduces double strand breaks (DSB). The DNA binding domain of a TALEN® is capable of targeting with high precision a large recognition site (for instance 17bp). Meganucleases are sequence-specific endonucleases, naturally occurring “DNA scissors”, originating from a variety of single-celled organisms such as bacteria, yeast, algae and some plant organelles. Meganucleases have long recognition sites of between 12 and 30 base pairs. The recognition site of natural meganucleases can be modified in order to target native genomic DNA sequences (such as endogenous genes).

Another recent genome editing technology is the CRISPR/Cas system, which can be used to achieve RNA-guided genome engineering. CRISPR interference is a genetic technique which allows for sequence-specific control of gene expression in prokaryotic and eukaryotic cells. It is based on the bacterial immune system-derived CRISPR (clustered regularly interspaced palindromic repeats) pathway.

Gene inactivation, i.e. inhibition of functional expression of the gene, may for instance also be achieved through the creation of transgenic organisms expressing antisense RNA, or by administering antisense RNA to the subject. An antisense construct can be delivered, for example, as an expression plasmid, which, when transcribed in the cell, produces RNA that is complementary to at least a unique portion of the cellular Slc7a11 RNA.

A more rapid method for the inhibition of gene expression is based on the use of shorter antisense oligomers consisting of DNA, or other synthetic structural types such as phosphorothioates, 2′-0-alkylribonucleotide chimeras, locked nucleic acid (LNA), peptide nucleic acid (PNA), or morpholinos. With the exception of RNA oligomers, PNAs and morpholinos, all other antisense oligomers act in eukaryotic cells through the mechanism of RNase H-mediated target cleavage. PNAs and morpholinos bind complementary DNA and RNA targets with high affinity and specificity, and thus act through a simple steric blockade of the RNA translational machinery, and appear to be completely resistant to nuclease attack. An “antisense oligomer” refers to an antisense molecule or anti-gene agent that comprises an oligomer of at least about 10 nucleotides in length. In embodiments an antisense oligomer comprises at least 15, 18 20, 25, 30, 35, 40, or 50 nucleotides. Antisense approaches involve the design of oligonucleotides (either DNA or RNA, or derivatives thereof) that are complementary to an RNA encoded by polynucleotide sequences of Slc7a11. Antisense RNA may be introduced into a cell to inhibit translation of a complementary mRNA by base pairing to it and physically obstructing the translation machinery. This effect is therefore stoichiometric. Absolute complementarity, although preferred, is not required. A sequence “complementary” to a portion of an RNA, as referred to herein, means a sequence having sufficient complementarity to be able to hybridize with the RNA, forming a stable duplex; in the case of double stranded antisense polynucleotide sequences, a single strand of the duplex DNA may thus be tested, or triplex formation may be assayed. The ability to hybridize will depend on both the degree of complementarity and the length of the antisense polynucleotide sequence. Generally, the longer the hybridizing polynucleotide sequence, the more base mismatches with an RNA it may contain and still form a stable duplex (or triplex, as the case may be). One skilled in the art can ascertain a tolerable degree of mismatch by use of standard procedures to determine the melting point of the hybridized complex. Antisense oligomers should be at least 10 nucleotides in length, and are preferably oligomers ranging from 15 to about 50 nucleotides in length. In certain embodiments, the oligomer is at least 15 nucleotides, at least 18 nucleotides, at least 20 nucleotides, at least 25 nucleotides, at least 30 nucleotides, at least 35 nucleotides, at least 40 nucleotides, or at least 50 nucleotides in length. A related method uses ribozymes instead of antisense RNA. Ribozymes are catalytic RNA molecules with enzyme-like cleavage properties that can be designed to target specific RNA sequences. Successful target gene inactivation, including temporally and tissue-specific gene inactivation, using ribozymes has been reported in mouse, zebrafish and fruit flies. RNA interference (RNAi) is a form of post-transcriptional gene silencing. The phenomenon of RNA interference was first observed and described in Caenorhabditis elegans where exogenous double-stranded RNA (dsRNA) was shown to specifically and potently disrupt the activity of genes containing homologous sequences through a mechanism that induces rapid degradation of the target RNA. Several reports describe the same catalytic phenomenon in other organisms, including experiments demonstrating spatial and/or temporal control of gene inactivation, including plant (Arabidopsis thaliana), protozoan (Trypanosoma brucei), invertebrate (Drosophila melanogaster), and vertebrate species (Danio rerio and Xenopus laevis). The mediators of sequence-specific messenger RNA degradation are small interfering RNAs (siRNAs) generated by ribonuclease III cleavage from longer dsRNAs. Generally, the length of siRNAs is between 20-25 nucleotides (Elbashir et al. (2001) Nature 411, 494 498). The siRNA typically comprise a sense RNA strand and a complementary antisense RNA strand annealed together by standard Watson Crick base pairing interactions (hereinafter “base paired”). The sense strand comprises a nucleic acid sequence that is identical to a target sequence contained within the target mRNA. The sense and antisense strands of the present siRNA can comprise two complementary, single stranded RNA molecules or can comprise a single molecule in which two complementary portions are base paired and are covalently linked by a single stranded “hairpin” area (often referred to as shRNA). The term “isolated” means altered or removed from the natural state through human intervention. For example, an siRNA naturally present in a living animal is not “isolated,” but a synthetic siRNA, or an siRNA partially or completely separated from the coexisting materials of its natural state is “isolated.” An isolated siRNA can exist in substantially purified form, or can exist in a non-native environment such as, for example, a cell into which the siRNA has been delivered.

The siRNAs of the invention can comprise partially purified RNA, substantially pure RNA, synthetic RNA, or recombinantly produced RNA, as well as altered RNA that differs from naturally occurring RNA by the addition, deletion, substitution and/or alteration of one or more nucleotides. Such alterations can include addition of non-nucleotide material, such as to the end(s) of the siRNA or to one or more internal nucleotides of the siRNA, including modifications that make the siRNA resistant to nuclease digestion.

One or both strands of the siRNA of the invention can also comprise a 3′ overhang. A “3′ overhang” refers to at least one unpaired nucleotide extending from the 3′ end of an RNA strand. Thus, in one embodiment, the siRNA of the invention comprises at least one 3′ overhang of from one to about six nucleotides (which includes ribonucleotides or deoxynucleotides) in length, preferably from one to about five nucleotides in length, more preferably from one to about four nucleotides in length, and particularly preferably from about one to about four nucleotides in length.

In the embodiment in which both strands of the siRNA molecule comprise a 3′ overhang, the length of the overhangs can be the same or different for each strand. In a most preferred embodiment, the 3′ overhang is present on both strands of the siRNA, and is two nucleotides in length. In order to enhance the stability of the present siRNAs, the 3′ overhangs can also be stabilized against degradation. In one embodiment, the overhangs are stabilized by including purine nucleotides, such as adenosine or guanosine nucleotides.

Alternatively, substitution of pyrimidine nucleotides by modified analogues, e.g., substitution of uridine nucleotides in the 3′ overhangs with 2′ deoxythymidine, is tolerated and does not affect the efficiency of RNAi degradation. In particular, the absence of a 2′ hydroxyl in the 2′ deoxythymidine significantly enhances the nuclease resistance of the 3′ overhang in tissue culture medium.

The siRNAs of the invention can be targeted to any stretch of approximately 19 to 25 contiguous nucleotides in any of the target Slc7a11 RNA sequences (the “target sequence”), of which examples are given in the application. Techniques for selecting target sequences for siRNA are well known in the art. Thus, the sense strand of the present siRNA comprises a nucleotide sequence identical to any contiguous stretch of about 19 to about 25 nucleotides in the target mRNA.

The siRNAs of the invention can be obtained using a number of techniques known to those of skill in the art. For example, the siRNAs can be chemically synthesized or recombinantly produced using methods known in the art. Preferably, the siRNA of the invention are chemically synthesized using appropriately protected ribonucleoside phosphoramidites and a conventional DNA/RNA synthesizer. The siRNA can be synthesized as two separate, complementary RNA molecules, or as a single RNA molecule with two complementary regions. Commercial suppliers of synthetic RNA molecules or synthesis reagents include Proligo (Hamburg, Germany), Dharmacon Research (Lafayette, Colo., USA), Pierce Chemical (part of Perbio Science, Rockford, III., USA), Glen Research (Sterling, Va., USA), ChemGenes (Ashland, Mass., USA) and Cruachem (Glasgow, UK).

Alternatively, siRNA can also be expressed from recombinant circular or linear DNA plasmids using any suitable promoter. Suitable promoters for expressing siRNA of the invention from a plasmid include, for example, the U6 or H1 RNA pol III promoter sequences and the cytomegalovirus promoter. Selection of other suitable promoters is within the skill in the art. The recombinant plasmids of the invention can also comprise inducible or regulatable promoters for expression of the siRNA in a particular tissue or in a particular intracellular environment. The siRNA expressed from recombinant plasmids can either be isolated from cultured cell expression systems by standard techniques, or can be expressed intracellularly, e.g. in breast tissue or in neurons.

The siRNAs of the invention can also be expressed intracellularly from recombinant viral vectors. The recombinant viral vectors comprise sequences encoding the siRNAs of the invention and any suitable promoter for expressing the siRNA sequences. Suitable promoters include, for example, the U6 or H1 RNA pol III promoter sequences and the cytomegalovirus promoter. Selection of other suitable promoters is within the skill in the art. The recombinant viral vectors of the invention can also comprise inducible or regulatable promoters for expression of the siRNA in the tissue where the tumour is localized.

As used herein, an “effective amount” of the siRNA is an amount sufficient to cause RNAi mediated degradation of the target mRNA, or an amount sufficient to inhibit the progression of metastasis in a subject. RNAi mediated degradation of the target mRNA can be detected by measuring levels of the target mRNA or protein in the cells of a subject, using standard techniques for isolating and quantifying mRNA or protein as described above.

One skilled in the art can readily determine an effective amount of the siRNA of the invention to be administered to a given subject, by taking into account factors such as the size and weight of the subject; the extent of the disease penetration; the age, health and sex of the subject; the route of administration; and whether the administration is regional or systemic. Generally, an effective amount of the siRNA of the invention comprises an intracellular concentration of from about 1 nanomolar (nM) to about 100 nM, preferably from about 2 nM to about 50 nM, more preferably from about 2.5 nM to about 10 nM. It is contemplated that greater or lesser amounts of siRNA can be administered.

Recently it has been shown that morpholino antisense oligonucleotides in zebrafish and frogs overcome the limitations of RNase H-competent antisense oligonucleotides, which include numerous non-specific effects due to the non-target-specific cleavage of other mRNA molecules caused by the low stringency requirements of RNase H. Morpholino oligomers therefore represent an important new class of antisense molecule. Oligomers of the invention may be synthesized by standard methods known in the art. As examples, phosphorothioate oligomers may be synthesized by the method of Stein et al. (1988) Nucleic Acids Res. 16, 3209 3021), methylphosphonate oligomers can be prepared by use of controlled pore glass polymer supports (Sarin et al. (1988) Proc. Natl. Acad. Sci. USA. 85, 7448-7451). Morpholino oligomers may be synthesized by the method of Summerton and Weller U.S. Pat. Nos. 5,217,866 and 5,185,444.

Another particularly form of antisense RNA strategy are gapmers. A gapmer is a chimeric antisense oligonucleotide that contains a central block of deoxynucleotide monomers sufficiently long to induce RNase H cleavage. The central block of a gapmer is flanked by blocks of 2′-O modified ribonucleotides or other artificially modified ribonucleotide monomers such as bridged nucleic acids (BNAs) that protect the internal block from nuclease degradation. Gapmers have been used to obtain RNase-H mediated cleavage of target RNAs, while reducing the number of phosphorothioate linkages. Phosphorothioates possess increased resistance to nucleases compared to unmodified DNA. However, they have several disadvantages. These include low binding capacity to complementary nucleic acids and non-specific binding to proteins that cause toxic side-effects limiting their applications. The occurrence of toxic side-effects together with non-specific binding causing off-target effects has stimulated the design of new artificial nucleic acids for the development of modified oligonucleotides that provide efficient and specific antisense activity in vivo without exhibiting toxic side-effects. By recruiting RNase H, gapmers selectively cleave the targeted oligonucleotide strand. The cleavage of this strand initiates an antisense effect. This approach has proven to be a powerful method in the inhibition of gene functions and is emerging as a popular approach for antisense therapeutics. Gapmers are offered commercially, e.g. LNA longRNA GapmeRs by Exiqon, or MOE gapmers by Isis pharmaceuticals. MOE gapmers or “2′MOE gapmers” are an antisense phosphorothioate oligonucleotide of 15-30 nucleotides wherein all of the backbone linkages are modified by adding a sulfur at the non-bridging oxygen (phosphorothioate) and a stretch of at least 10 consecutive nucleotides remain unmodified (deoxy sugars) and the remaining nucleotides contain an O′-methyl O′-ethyl substitution at the 2′ position (MOE).

### Antibodies Inhibiting the Activity of Slc7A11

Antibodies specifically binding to Slc7a1, particularly antibodies binding to an extracellular region of Slc7a11 can inhibit the activity of Slc7a11 and such antibodies are for use to treat wound healing, particularly chronic wound healing in a mammal.

The term “antibody” as used herein, refers to an immunoglobulin (Ig) molecule or a molecule comprising an immunoglobulin (Ig) domain, which specifically binds to Slc7a11, preferably with an extracellular domain of Slc7a11. ‘Antibodies’ can further be intact immunoglobulins derived from natural sources or from recombinant sources and can be immunoreactive portions of intact immunoglobulins. The term “active antibody fragment” refers to a portion of any antibody or antibody-like structure that by itself has high affinity for an antigenic determinant, or epitope, and contains one or more CDRs accounting for such specificity. Non-limiting examples include immunoglobulin domains, Fab, F(ab)′2, scFv, heavy-light chain dimers, immunoglobulin single variable domains, Nanobodies, domain antibodies, and single chain structures, such as a complete light chain or complete heavy chain. Examples of antibodies specifically binding to an extracellular epitope of Slc7a11 and inhibiting the activity of Slc7all are taught in WO2018/204278 and WO2020/227640.

### Combinations

In a particular embodiment a combination of an inhibitor of Slc7a11 and other molecules for use to treat wound healing, particularly chronic wound healing is envisaged.

Even though inhibition of Slc7a11 is sufficient to achieve a therapeutic effect, i.e. to achieve wound healing, particularly chronic wound healing, it is shown herein in the appended example section that a stronger, synergistic effect is achieved when both an inhibitor of Slc7a11 and the growth factor GDF-15 are administered. The synergistic effect can be obtained through simultaneous, concurrent, separate or sequential use for treating wound healing, particularly chronic wound healing.

In yet another embodiment the invention provides a composition consisting of an inhibitor of Slc7a11 and GDF-15 for use as a medicament.

In yet another embodiment the invention provides a composition consisting of an inhibitor of Slc7a11 and GDF-15 for use to treat wound healing, particularly chronic wound healing in a mammal.

### Pharmaceutical Compositions

This invention also relates to pharmaceutical compositions containing one or more compounds of the present invention for use to treat wound healing, particularly chronic wound healing. These compositions can be utilized to achieve the desired pharmacological effect by administration to a mammal, such as a patient in need thereof. A patient, for the purpose of this invention, is a mammal, including a human, in need of treatment for the particular condition or disease. Therefore, the present invention includes pharmaceutical compositions that are comprised of a pharmaceutically acceptable carrier and a pharmaceutically effective amount of a compound, or salt thereof, of the present invention for use to treat wound healing, particularly chronic wound healing. A pharmaceutically acceptable carrier is preferably a carrier that is relatively non-toxic and innocuous to a patient at concentrations consistent with effective activity of the active ingredient so that any side effects ascribable to the carrier do not vitiate the beneficial effects of the active ingredient. A pharmaceutically effective amount of compound is preferably that amount which produces a result or exerts an influence on the particular condition being treated. The compounds of the present invention can be administered with pharmaceutically-acceptable carriers well known in the art using any effective conventional dosage unit forms, including immediate, slow and timed release preparations, orally, parenterally, topically, nasally, ophthalmically, optically, sublingually, rectally, vaginally, and the like.

In a particularly preferred embodiment the administration is topical administration.

Another formulation which can be employed in the present invention are transdermal delivery devices (“patches”). Such transdermal patches may be used to provide continuous or discontinuous infusion of one or more inhibitors of Slc7a11 in controlled amounts. The construction and use of transdermal patches for the delivery of pharmaceutical agents is well known in the art (see for example U.S. Pat. No. 5,023,252). Such patches may be constructed for continuous, pulsatile, or on demand delivery of pharmaceutical agents.

### Dose and Administration

Based upon standard laboratory techniques known to evaluate compounds useful for the treatment of diseases cited herein, by standard toxicity tests and by standard pharmacological assays for the determination of treatment of the conditions identified above in mammals, and by comparison of these results with the results of known medicaments that are used to treat these conditions, the effective dosage of the compounds of this invention can readily be determined for treatment of the indications cited herein. The amount of the active ingredient to be administered in the treatment can vary widely according to such considerations as the particular compound and dosage unit employed, the mode of administration, the period of treatment, the age and sex of the patient treated, and the nature and extent of the condition treated.

The total amount of the active ingredient to be administered will generally range from about 0.001 mg/kg to about 200 mg/kg body weight per day, and preferably from about 0.01 mg/kg to about 50 mg/kg body weight per day. Clinically useful dosing schedules will range from one to three times a day dosing to once every four weeks dosing. In addition, “drug holidays” in which a patient is not dosed with a drug for a certain period of time, may be beneficial to the overall balance between pharmacological effect and tolerability. A unit dosage may contain from about 0.5 mg to about 1500 mg of active ingredient, and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight. The average daily oral dosage regimen will preferably be from 0.01 to 100 mg/kg of total body weight.

It is evident for the skilled artisan that the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

It is to be understood that although particular embodiments, specific configurations as well as materials and/or molecules, have been discussed herein for cells and methods according to the present invention, various changes or modifications in form and detail may be made without departing from the scope and spirit of this invention. The following examples are provided to better illustrate particular embodiments, and they should not be considered limiting the application. The application is limited only by the claims.

### EXAMPLES

### 1.Upregulation of Amino Acid Transporters in Dendritic Cells Engulfing Apoptotic Cells

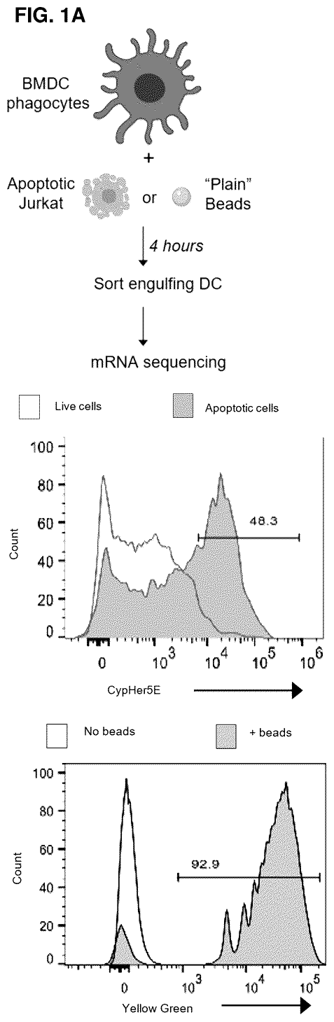

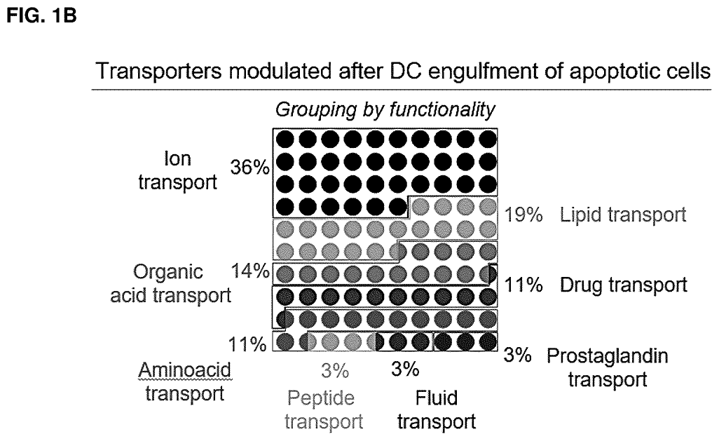

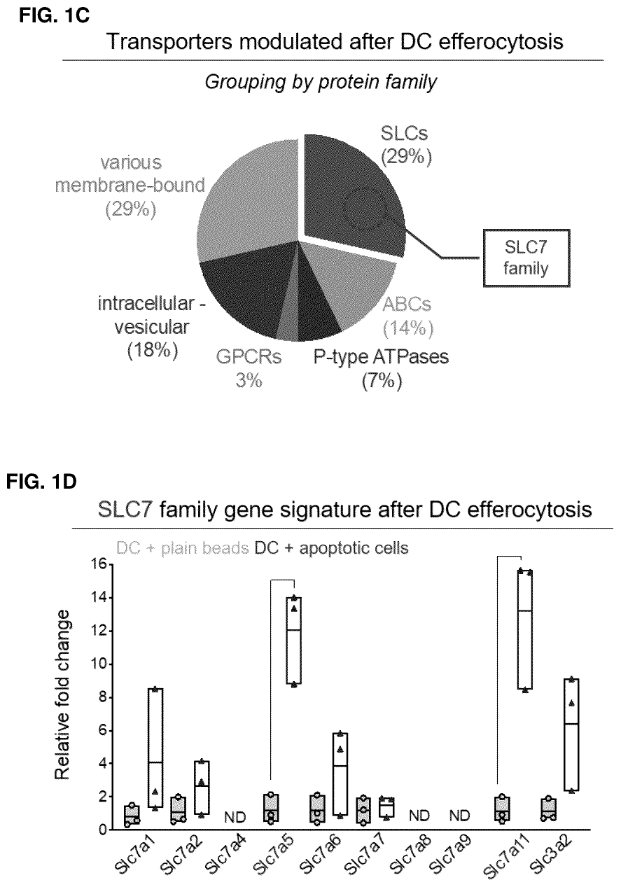

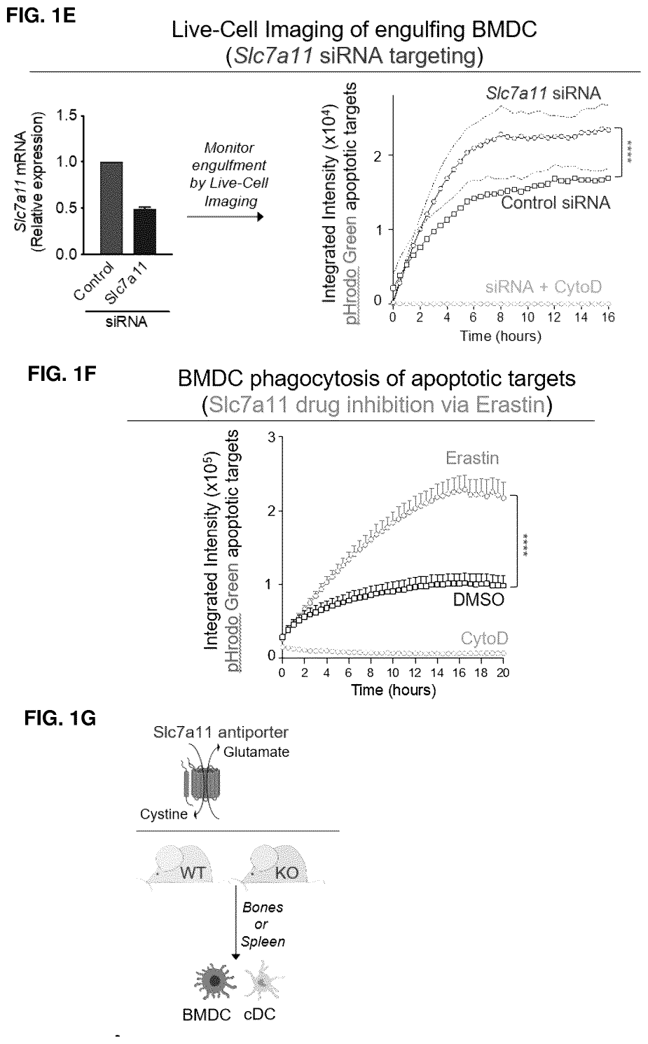

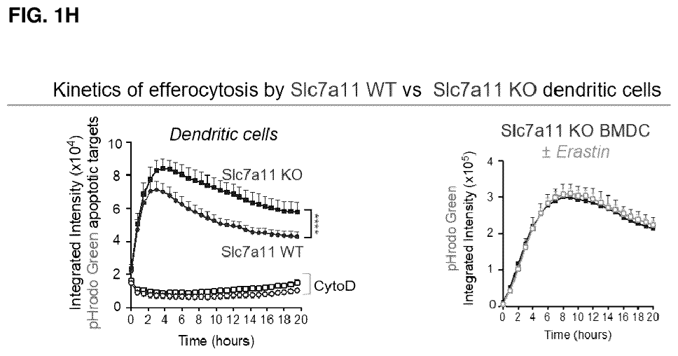

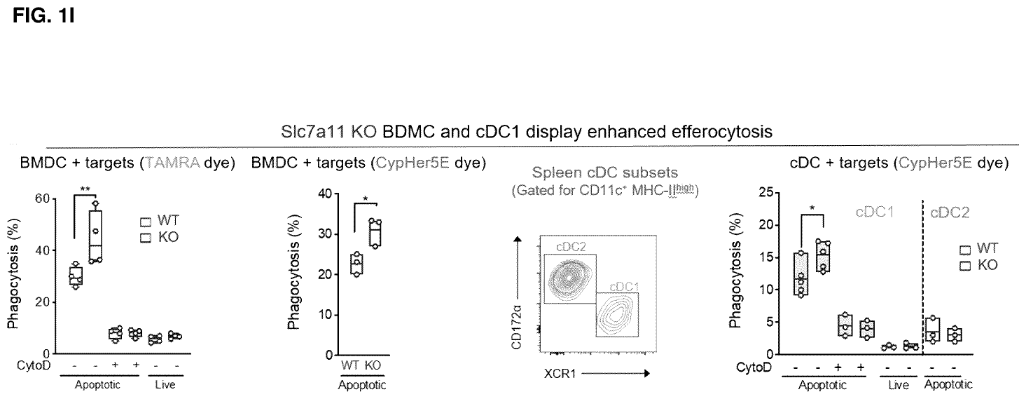

Gene signatures initiated in dendritic cells during efferocytosis are not yet defined, and adaptation of dendritic cells to the metabolic challenge of ingesting cargo remain unclear. To address this, we incubated primary mouse bone-marrow dendritic cells (BMDC phagocytes) with apoptotic human Jurkat cells, purified the efferocytic dendritic cells and performed RNAseq analysis (FIG. 1a). We used this cross-species approach to easily distinguish the mouse phagocytic RNA from any apoptotic cargo-derived RNA. We also used plain beads as targets to control for phagocytosis. In our analysis, we focused on differentially expressed genes associated with ‘transporter’ activity based on the initial hypothesis that dendritic cells may have molecular features that differ from other phagocytes on how they perceive and interpret the ingested apoptotic cargo. We found significant alterations in genes encoding membrane transporters with diverse functionalities including those that passage ions, lipids, and amino acids (FIG. 1b). Interestingly, the solute carrier (Slc) family of genes represented 29% of the differentially expressed transporters in efferocytic dendritic cells (FIG. 1c). SLCs represent the second largest gene family in the human genome (after the GPCRs) and are comprised of membrane proteins that mediate transport of metabolites and solutes across cellular membranes. SLCs are linked to >100 human diseases, are amenable for targeting by small molecules, and have been implicated in efferocytosis by other phagocytes²¹ (FIG. 1c). Among the SLCs, those coding for amino acid metabolism and carbohydrate catabolismwere upregulated in efferocytic dendritic cells, while SLCs linked to oxidative phosphorylation (OXPHOS) and fatty acid transport were downregulated (FIG. 5a).

Amino acid transport is fundamental for nutrient supply and supports key functions of immune cells^{22,23}, including dendritic cells²⁴. Thus, we chose to focus on several Slc7 family members. We examined cationic (Slc7a1through Slc7a4) and hetero(di)meric amino acid transporters (Slc7a5 through Slc7a11), as well as Slc3a2, which helps to chaperone/facilitate cell membrane localization of some Slc7 members²⁵. Among the Slc7 family members, efferocytic dendritic cells had the greatest increase in Slc7a5 and Slc7a11 (FIG. 1d).

### 2.Slc7a11 Antiporter Acts as a Brake for Efferocytosis by Dendritic Cells

To test the relevance of these amino acid transporters upregulated in efferocytic BMDC, we performed siRNA-mediated knockdown in BMDCs, followed by efferocytosis via a flow cytometry-based assay and an independent live cell imaging assay for apoptotic cell uptake over a time course. Among the Slc7 family members that we targeted via siRNA, interfering with Slc7a11 expression consistently caused a significant increase in efferocytosis by dendritic cells (FIG. 1e). Slc7a11, also known in the literature as xCT, is the specific subunit of the cystine-glutamate antiporter system x, that regulates the exchange of intracellular glutamate for extracellular cystine, the latter being a rate-limiting precursor for the synthesis of antioxidant glutathione (GSH). Slc7a11 is also linked to neurological diseases^{26-28,29,30}, viral infections^{31,32,33} and multiple cancers^{34,35,36}, with drugs targeting Slc7a11 tested in clinical trials. Under these conditions, knockdown of Slc71 did not have an impact on BMDC efferocytosis, while knockdown or pharmacologic inhibition of Slc7a5 only showed a trend toward more efferocytosis but was not statistically significant (FIG. 5b-e). We were initially surprised that Slc7a11 acts as a negative regulator of efferocytosis, as its expression goes up robustly during efferocytosis. Therefore, we took additional pharmacological and genetic approaches to address the role of Slc7a11 in dendritic cell efferocytosis. Inhibition of Slc7a11 with the drug erastin³⁷ caused a dramatic boost in efferocytosis (FIG. 1f); in contrast, erastin did not affect the phagocytosis of E. coli bioparticles by dendritic cells (FIG. 6a). Although erastin has been described as a ferroptosis inducer in cancer cells^{38,39,40}, the enhanced efferocytosis in dendritic cells was not linked to ferroptosis, as another known ferroptosis-inducing compound, ML-162³⁸ did not enhance efferocytosis (FIG. 6b). While erastin treatment did reduce intracellular glutathione levels (FIG. 6c), it only modestly increased lipid ROS accumulation (FIG. 6e) and did not affect dendritic cell viability (FIG. 6d). The effect of erastin can also be mimicked by adding excess glutamate to ‘block’ the cystine uptake via Slc7a11, and this again boosted efferocytosis (Extended FIG. 2f).

As a genetic approach, we tested different types of dendritic cells from Slc7a11-deficient mice. Comparing purified primary splenic conventional dendritic cells from control and Slc7a11-deficient mice, we noted enhanced uptake by Slc7a11-deficient cDC1, but not cDC2. Similarly, ex vivo generated BMDC lacking Slc7a11 displayed greater efferocytosis compared to controls (FIG. 1g, h, i). This enhanced efferocytosis by Slc7a11-deficient dendritic cells was seen using apoptotic targets labelled with either the pH-insensitive (TAMRA) or pH-sensitive (CypHer5E) reporter dyes (FIG. 1i), and this was abrogated by blocking cytoskeletal rearrangement with cytochalasin D (FIG. 1h, i). The increased uptake scored in these assays was not due to defective digestion of apoptotic corpses, as Slc7a11-deficient dendritic cells showed similar corpse degradation as wild type dendritic cells (measured by CellTrace Violet labeling of apoptotic cells) (FIG. 5f). Supporting the specificity for the effect of erastin on Slc7a11 in this assay, there was no additive increase in efferocytic capacity of Slc7a11-deficient dendritic cells treated with erastin (FIG. 1h). Interestingly, neither bone marrow-derived macrophages from Slc7a11-deficient mice, nor peritoneal macrophages from wild-type mice treated with erastin showed enhanced efferocytosis under these conditions (FIG. 5g-h). Lastly, the Slc7a11-deficient dendritic cells did not show an enhanced uptake of ‘live’ cells (FIG. 1i). These data collectively suggest that Slc7a11 acts as brake on dendritic cell efferocytosis.

### 3.Skin Wound Healing is Accelerated in the Context of Slc7a11 Blockade

As the presence of uncleared apoptotic cells is often linked to chronic inflammation⁴¹, we wished to address the physiological relevance of the enhanced efferocytosis due to loss or inhibition of Slc7a11. We first assessed gene expression profiles of SLCs in resident dendritic cell subsets in the spleen, lung, or the skin. Mining publicly available datasets (ImmGen) and via Tri-wise comparison, Slc7a11 gene is most highly expressed in skin-resident dendritic cells compared to DCs from spleen or lung (FIG. 2a). In contrast, Slc705 is more expressed in the spleen and lung DCs. Previous studies have suggested that depletion of Langerin⁺ cells (located in the epidermis) enhances cutaneous wound healing⁴², while depletion of dermal CD11c⁺ dendritic cells (located in the dermis) delays wound closure⁴³. Hence, we next addressed the link between Slc7a11 and dendritic cells during skin regeneration. When we tested purified dermal dendritic cells isolated from the ears of Slc7a11-deficient mice, the cDC1 cells showed enhanced efferocytosis compared to those from wild-type mice; in comparison, Langerhans cells and cDC2 were poor engulfers ex vivo and their phagocytic capacity was unaltered by Slc7a11-deficiency (FIG. 2b, FIG. 7a). While CD64^{pos} macrophages were potent at apoptotic cell uptake, Slc7a11 deficiency did not modify their uptake (FIG. 2b). In histological analysis of wounded skin tissue biopsies, Slc7a11 was colocalized with the DC marker CD11c (FIG. 2c), but not with fibroblasts (FIG. 7b). Additionally, CD11c⁺ dendritic cells were proximal to and appeared to capture apoptotic corpses, as shown by cleaved caspase-3⁺ staining (FIG. 2d). We also noted an increase in Slc7a11 expression in total skin lysates after full-thickness wounding, along with increase in expression of genes linked to wounding such as Tgfb1 and Tnf (FIG. 2f).

Next, we tested whether interfering with Slc7a11 function may impact wound healing dynamics in the skin. We topically administered a metabolically stable and water soluble version of erastin (called IKE)^{45,46} to mice after full-thickness skin wounding (on day 0 to day 2). However, administering erastin alone to inhibit Slc7a11 at the wound site did not improve wound healing (FIG. 7f). As previous work from many groups have suggested that the efferocytic phagocytes provide many beneficial factors in dampening inflammation^{2,3,4,5}, we asked whether co-administering some early stage apoptotic cells along with the erastin might be of benefit. Remarkably, single administration to the wound site of apoptotic cells at day 0 (in the form of apoptotic Jurkat cells) along with erastin at day 0 and day 2 (referred to as ‘erastin regimen’ from here onwards) markedly accelerated the wound healing (FIG. 2g, FIG. 7e), resulting in faster and complete closure of initial wounds (FIG. 2h). The wound closure in with erastin-treated mice reached 50% of wound closure at day 4 post wounding, nearly 2 full days earlier than their respective controls (FIG. 2g). Importantly, administering apoptotic cells alone without erastin did not improve the dynamics of wound closure (FIG. 7f). Slc7a11 inhibition via the erastin regimen greatly decreased the apoptotic cell burden in the wound, as evidenced by fewer cells that stained positive for cleaved caspase-3 (FIG. 2i). Thus, the accelerated wound healing requires the combination of a bolus of early-stage apoptotic cells together with Slc7a11 inhibition via erastin, the latter to improve efferocytosis at the wound site. The benefit of this erastin regimen was not related to induction of ferroptosis in the skin, as a ferroptosis inducer RSL3^{40,47} (given together with apoptotic cells) did not improve wound healing (FIG. 7g). To test the erastin regimen on existing wounds, we administered erastin on day 2 to day 4 post-wounding, which also led to faster healing after skin wounding (FIG. 2j).

To complement the above studies with a genetic approach, we tested Slc7a11-deficient mice (FIG. 2k). Slc7a11-deficient mice also showed accelerated wound healing dynamics, and this also required co-administration of apoptotic cells on day 0 post-wounding. The wound closure in the Slc7a11-null mice also reached 50% of wound closure nearly 2 days earlier than the respective controls (FIGS. 2g and 2k). These data strongly suggest that interfering with Slc7a11 function in the presence of apoptotic cells can accelerate wound healing in the skin.

### 4.Glycogen Reserves and Aerobic Glycolysis Fuel Enhanced Efferocytosis in Dendritic Cells

We were intrigued by the notion that while the combination of Slc7a11 inhibition together with apoptotic cells accelerated cutaneous wound healing, neither of them alone had much of a significant effect. To better understand how loss/inhibition of Slc7a11 synergizes with apoptotic cells, we performed RNAseq analysis of control and Slc7a11-deficient dendritic cells engulfing apoptotic cells (FIG. 3a). While there was very little basal difference in gene expression between WT and Slc7a11 KO dendritic cells, efferocytic Slc7a11-deficient dendritic cells had 191 differentially expressed genes (125 upregulated and 66 downregulated) compared to control efferocytic dendritic cells. Literature analyses of these genes for their association with specific pathways indicated that the differentially expressed genes represented biological processes, including metabolism, protein synthesis, mitochondrial and ER function, transcriptional regulation, and wound healing (FIG. 3b, FIG. 8a). Metabolic function was the most represented transcriptional program initiated in Slc7a11-deficient compared to WT efferocytic dendritic cells and encompassed genes associated with gluconeogenesis (Gpt2, Dyrk1b, Hlcs, Pck2)^{48,49}, diabetes and obesity (Gdf15, Pck2)^{50,51,52}, amino acid synthesis (Asns, Phgdh), glutamate metabolism (Aldh18a1, Psat1, Gpt2, Got1), and lipid metabolism (Cyb5r1, Soat2, Acaa1b, Cd5l).

Previously, it has been shown in macrophages that aerobic glycolysis of glucose is critical for the initial uptake as well as the continued uptake of additional apoptotic corpses²². Dendritic cells have a unique feature among phagocytes in that they possess intracellular glycogen reserves⁵³, and interestingly, genes related to gluconeogenesis (i.e. generation of glucose) came up as one of the top hits in the RNAseq of Slc7a11-deficient efferocytic dendritic cells compared to WT dendritic cells. Thus, we considered an exciting possibility that the Slc7a11-deficient dendritic cells might have increased capacity to convert some of their glycogen reserves to generate more glucose, which may in turn be used for aerobic glycolysis, and to achieve increased efferocytosis. We first assessed whether glycogen reserves differ between control and Slc7a11-deficient dendritic cells, as well as WT dendritic cells before and after erastin treatment. Both Slc7a11-deficient BMDC as well as erastin-treated wild type cDC1 showed reduced intracellular glycogen (FIG. 3c). Importantly, addition of the glycogen phosphorylase inhibitor CP-91149 reversed the glycogen levels in erastin-treated BMDC almost to DMSO control (FIG. 3d). We then asked whether glycogen breakdown was important for the increased efferocytosis after Slc7al1 inhibition. Addition of CP-91149 strongly abrogated the erastin-dependent increase in efferocytosis (FIG. 3d), supporting the notion that glycogenolysis contributes to greater DC efferocytosis.

Next, we asked whether aerobic glycolysis was important for dendritic cells efferocytosis. In Seahorse analysis, Slc7a11-deficient or erastin treated dendritic cells showed greater aerobic glycolysis (ECAR) and decreased oxygen consumption rate (OCR, suggestive of mitochondrial oxidative phosphorylation), along with a significantly reduced spare respiratory capacity (FIG. 3e and FIG. 9a). During efferocytosis, both WT and erastin-treated dendritic cells showed greater aerobic glycolysis (ECAR) and less OCR (FIG. 3e). To test whether aerobic glycolysis plays a role in the enhanced efferocytosis by Slc7a11-deficient dendritic cells, we used several pharmacological inhibitors. First, a non-metabolizable glucose analogue 2-deoxyglucose (2-DG) abrogated the enhanced efferocytosis due to Slc7a11 inhibition (FIG. 3f). Further, a cell-permeable pyruvic-acid derivative 3-bromopyruvate (3-BP), acting as an inhibitor of hexokinase-II mediated glycolysis (FIG. 3f), also dampened the enhanced inhibition due to erastin treatment. When we asked if alternative fuel substrates such as glutamine or pyruvate could be used by Slc7a11-inhibited dendritic cells, this did not appear to be the case: a glutamine antagonist (DON) marginally decreased the erastin-mediated augmented efferocytosis, while inhibiting the import of pyruvate into the mitochondria (via the drug UK5099) had no impact on efferocytosis (FIG. 9c). Of note, none of the inhibitors interfered with dendritic cell viability (FIG. 9b). Collectively, these data suggest that conversion of glycogen stores in dendritic cells to glucose and the subsequent aerobic glycolysis contribute to the enhanced efferocytosis by Slc7a11-deficient dendritic cells.

### 5.Slc7a11 inhibition and rGDF15 facilitate wound healing in diabetic mice

Non-healing chronic wounds are a serious complication that significantly impacts the quality of life in diabetic patients54. To test the relevance of Slc7a11 on wound healing in the diabetic context, we used leptin receptor-deficient db/db mice, which represent a model of human type 2 diabetes with obesity and hyperglycemia. We first confirmed that wound healing is impaired in db/db mice in the skin excision model; healing of wounds in the db/db mice was delayed by nearly 3 days to reach half-maximal closure (FIG. 4a, and ref 9%). When we examined the level of Slc7a11 expression, strikingly, it was 200-fold higher on day 4 in the wounds of db/db mice compared to normoglycemic C57B/6 mice (FIG. 4b). We then tested whether Slc7a11 inhibition might affect diabetic wound healing, by treating db/db mice post-wounding with erastin alone as well as with the erastin regimen (with apoptotic cell co-administration). Unlike non-diabetic wounds, diabetic wounds treated only with erastin displayed acceleration of closure, likely because there are enough uncleared apoptotic cells in the diabetic wounds (FIG. 4c). Indeed, while the wounds of C57B/6 mice reached a mean of 400 apoptotic cells (cleaved caspase 3+) at day 4 (FIG. 2i) and dropped to 50 at day 13, the diabetic wounds still scored a mean of 400 at day 13 (FIG. 4c) reflecting the increased apoptotic load. The erastin regimen with apoptotic cells also provided significant improvement as early as day 2 after wounding, and this continued through full closure by day 13. Notably, the wounds of db/db mice treated via the erastin regimen reached 50% closure of initial wound 2-3 days earlier than control mice (FIG. 4d).

Next, we tested the link between apoptotic cell clearance and the accelerated wound closure due to erastin treatment in the db/db mice. In ex vivo analysis, dendritic cells from diabetic mice showed reduced efferocytosis compared to wild-type controls with the difference being more pronounced at earlier time points (FIG. 4e), akin to the defective efferocytosis seen by macrophages from diabetic mice¹³. Strikingly, erastin treatment ‘rescued’ the defective efferocytosis of diabetic db/db dendritic cells (FIG. 4e), to the level of wild-type dendritic cells treated with erastin. Consistent with this ex vivo phenotype, the erastin regimen substantially reduced the number of uncleared cleaved caspase 3⁺ apoptotic cells in the wounds of db/db mice (FIG. 4f).

As the combination of Slc7a11 inhibition together with apoptotic cell addition to the wounds provided the most efficient wound healing, and efferocytic phagocytes also secrete factors that are known to provide beneficial effects in tissues56, we asked whether secreted factors from efferocytic Slc7a11-deficient dendritic cells may contribute to the accelerated healing. We purified supernatants from efferocytic wild type and Slc7a11 KO dendritic cells and added these exogenous supernatants to the excision wounds. Mice treated with efferocytic supernatants from efferocytic Slc7a11-deficient dendritic cells exhibited faster wound closure compared to WT dendritic cells (FIG. 4g).

Soluble factors including members of TGF-β superfamily (TGF-β 1-3) are known to be produced by efferocytic phagocytes and play important roles during different stages of wound healing⁵⁷. When we mined the transcriptome of efferocytic Slc7a1-deficient dendritic cells for TGF-β superfamily members, we noted that Gdf15 was specifically upregulated (FIG. 4h). GDF15 was also significantly increased in the supernatants of efferocytic dendritic cells from Slc7a11-deficient mice compared to that of wild-type littermates (FIG. 4h). Growth differentiation factor-15 (GDF15) is a recently discovered member of the transforming growth factor B (TGF-β) superfamily that has been associated with obesity, diabetes, cancer, cardiovascular and kidney disorders^{50,58,59}. During cutaneous wound healing, the GDF15 levels were increased (2-5 fold) at day 2 post excisional skin wounding compared to unwounded skin and remained high even at later stages of the healing process (day 8) (FIG. 4i). We next tested GDF15-deficient mice in the excisional skin biopsy model. GDF15 knockout mice displayed larger wounds at day 2 compared to littermate control mice (FIG. 4j), and this correlated with greater Slc7a11 expression in the wounds (FIG. 4b). Consistent with the notion that GDF15 is produced downstream efferocytosis, GDF15-deficient dendritic cells showed comparable efferocytosis as controls (FIG. 10a). We next asked whether addition of recombinant GDF15 (rGDF15) might also help wound healing in the diabetic db/db mice. Indeed, application of rGDF15 significantly improved wound healing in the db/db mice (FIG. 4k). Together, these data suggest that Slc7a11 inhibition at the wound site can improve diabetic wound healing via at least two modalities: by reversing the impaired cell clearance by dendritic cells and through secretion of factors such as GDF15 by the efferocytic cells that promote wound healing.

### 6.Discussion

The data presented in this invention, using a combination of in vitro, ex vivo, and in vivo approaches, advance new concepts related to dendritic cell-mediated efferocytosis and its relevance to tissue regeneration/wound healing. First, we demonstrate that expression of multiple membrane proteins and transporters are modulated during apoptotic cell engulfment by dendritic cells. Among these membrane proteins, the amino acid transporter Slc7a11 acts as a negative regulator of efferocytosis. As inhibition or loss of Slc7a11 leads to improved wound healing in both non-diabetic and diabetic mice, it is unclear why this negative regulator of efferocytosis would be upregulated following wounding. Nevertheless, similar concurrent upregulation of negative and positive regulators (i.e. the simultaneous pressing of the ‘accelerator’ and the ‘brake’) has been seen in T cells during antigen receptor signaling, in fibroblasts during growth factor stimulation, and in macrophages during FcR-mediated phagocytosis, and is likely part of a finely-balanced signaling regulation in vivo. Mechanistically, the enhanced efferocytosis in Slc7a11-deficient dendritic cells is in part fueled by greater aerobic glycolysis, where the source of glucose is derived from glycogen stores, a feature somewhat unique to dendritic cells among phagocytes.

This invention also reveals dendritic cells as relevant players in clearing apoptotic cells during cutaneous skin injury. Most strikingly, the combination of Slc7a11 blockade together with apoptotic cells can greatly improve the wound healing kinetics. This accelerated wound healing is not only seen in wild type mice, but more importantly in diabetic prone db/db mice. While anti-inflammatory soluble factors are known to be released from efferocytic phagocytes, transcriptomics analysis of Slc7a11-deficient dendritic cells identified GDF15 as one key extracellular mediator that facilitates wound closure downstream of Slc7a11. Thus, inhibition of Slc7a11 function as well as addition of certain soluble downstream factors (e.g. GDF15) can improve cutaneous wound healing in diabetic conditions.

Taken together, this invention, starting from the unbiased transcriptomics of efferocytic dendritic cells to identifying Slc7a11 as a brake on dendritic cell phagocytosis and mechanistic studies, reveals a new approach to improve cutaneous wound healing. This strategy is relevant for chronic wound healing management such as diabetic wound healing.

### Methods

Murine tissue processing. Cells from ears of Slc7a11 KO and control littermates were isolated as previously described⁶⁰. Briefly, ear skin samples were collected by cutting at the ear base and were incubated overnight at 4° C. with 200 μg/ml Dispase Il (from Bacillus polymerase grade 2; Roche, Basel, Switzerland) to facilitate manual cutting and isolation of cells. Small skin pieces were further digested with 1.5 mg/ml collagenase type 4 (Worthington, Lakewood, NJ) and 10 U DNase (Roche) in RPMI medium buffered with HEPES and supplemented with 2% fetal calf serum. The suspension was resuspended every 30 minutes and provided with fresh digestion buffer for a total of 90 minutes at 37° C. After digestion, the cell suspension was filtered to remove debris and clots. For spleen single-cell suspensions, spleens were digested in RPMI medium supplemented with 0.01 U/ml DNase I (Roche) and 0.02 mg/ml Liberase (Roche) for 30 min. Red blood cells were removed with 1× solution of RBC lysis buffer (Biolegend; #420301). For bone marrow progenitors, bone marrow was isolated from femurs and tibia of 8- to 12-week-old mice.

Cell isolation. Single-cell suspensions of digested ears and spleens were further enriched for phagocytes by depleting lymphocyte populations. Depletion was performed using monoclonal biotin-linked antibodies against CD3e (145-2C11, #13-0031-82 eBioscience™), CD19 (eBio1D3-1D3, #13-0193-82 eBioscience™), NK1.1 (PK136, #13-5941-82 eBioscience™) followed by collection of non-depleted cells using MagniSort™ Streptavidin Negative Selection Beads (#MSNB-6002-74 ThermoFisher). Bone-marrow-derived dendritic cells and macrophages were generated from mice by culturing bone marrow progenitors for 10 days in GM-CSF-supplemented medium and for 6 days in M-CSF-supplemented medium respectively and as previously described⁶¹.

Flow cytometry and cell sorting. Immunophenotyping of mouse skin or spleen was performed on single-cell suspensions. Cells were stained with the following anti-mouse, monoclonal, fluorochrome-linked antibodies: CD24-AF488 or-eFluor450 (M1/69; #101816 or #48-0242-82), CD11b-BV605 (M1/70; #563015), CD26-FITC or-BV650 (H194-112; #559652 or #740474), CD11c-BV711 (HL3; #563048), F4/80-BV785 (BM8; #123141), CD45-AF700 (30-F11; #56-0451-82), MHCII (I-A/I-E)-eFluor780 (M5/114.15.2; #47-5321-82), CD172a (SIRP alpha)-PerCP-eFluor710 (P84; #46-1721-82), CD103-BUV395 (M290; #740238), CD64-BV421 or-PE/Cy7 (X54-5/7.1; #139309 or #139314), XCR1-BV650 (ZET; #148220) and Fc receptor-blocking antibody CD16/CD32 (clone 2.4G2, #553142). Viable cells were discriminated using Fixable Viability Dye eFluor 506 (#65-0866-18). Prior to measuring, counting beads (#01-1234-42) were added to the cells for some experiments. Measurements were performed on a BD LSR Fortessa cytometer and analyzed using FlowJo10 software (Tree Star). Cell sorting was performed on FACS ARIAII and III (BD Biosciences).

Ferroptosis characterization. To assess ferroptosis, we utilized BODIPY™ 581/591 (C11-BODIPY) and dihydrorhodamine 123 (DHR123) probes that change their fluorescence properties upon oxidation, as previously described⁶². Briefly, dendritic cells were treated with 5 μM erastin (#S7242, Bio-Connect B.V.) or 1 μM ML-162 (#AOB1514) or DMSO control prior to addition of fluorescent probes one hour before measurement: 0.5 μM C11-BODIPY (#D3861, ThermoFisher) or 1 M DHR-123 (#85100, Chemical, MI, USA) and 0.5 μM of DRAQ7 cell death stain (#DR70250, BioStatus, Shepshed, UK).

Determination of glycogen concentration. The glycogen concentration was measured using a Glycogen Colorimetric/Fluorometric Assay Kit (#GENT-K646-10, BioVision) according to the manufacturer's instructions.

Determination of glutathione levels. Glutathione was quantified in dendritic cells two hours after erastin treatment using the GSH/GSSG-Glo Assay luminescence-based system according to the manufacturer's instructions (#V6611, Promega). GSH/GSSG ratios are calculated from luminescence measurements (in relative light units, RLU) and after interpolation of glutathione concentrations from standard curves. Data for both glycogen and glutathione are reported as fold change from DMSO (vehicle)-treated cells or from WT cells.

Seahorse analysis. 100000 BMDC were seeded on a Seahorse 96-well tissue culture plate (Agilent Technologies). The plate was allowed to stand for 30 mins for the cells to settle before placing it in the incubator overnight. The adhered cells were treated with 5 μM erastin (#57242, Bio-Connect B.V) or 200 μM CP-91149 (#S2717, Bio-Connect B.V.) 2 hours prior to Seahorse analysis. The cells were switched to serum-free Seahorse media before the assay according to the manufacturer's instructions. For basal ECAR and OCR, the cells were subjected to XF Real Time ATP Rate kit (#103592-100, Agilent Technologies). For assessment of respiratory capacity, cells were subjected to XF Cell Mito Stress Test Kit (#103015-100, Agilent Technologies). The sequential injection of Oligomycin, FCCP, and rotenone/Antimycin A were done at 1.5 μM, 1.0 μM and 0.5 μM respectively.

Efferocytosis assays. For induction of apoptosis, human Jurkat T cells were with stained with CypHerSE (#PA15401, GE Healthcare) or TAMRA (#C-1171, Invitrogen) or pHrodo™ Green STP Ester (#P35369, ThermoFisher), resuspended in RPMI with 5% fetal calf serum, treated with 150 mJ/cm² ultraviolet C irradiation (Stratalinker) and incubated for 4 h at 37° C. with 5% CO₂. Dendritic cells were incubated with apoptotic targets at a 1:5 phagocyte: target ratio for the indicated times. Phagocytosis was assessed by a flow cytometry-based assay²¹ or by Incucyte Live-cell imaging. As alternative targets for phagocytosis, dendritic cells were incubated with pHrodo™ Green E. coli BioParticles™ (#P35366, ThermoFisher) or Streptavidin Fluoresbrite® YG Microspheres, 2.0 μm (for simplicity, beads) at a 1:5 phagocyte: target ratio for the indicated times. When applicable, cells were pretreated for 1 hour with 5 μM erastin (#S7242, Bio-Connect B.V), 200 μM CP-91149 (#S2717, Bio-Connect B.V.), 0.2 mM 2-DG (2-Deoxy-D-glucose; #D8375-1G, Sigma), 5 μM UK5099 (#PZ0160, Sigma), 40 μM DON (6-Diazo-5-oxo-L-norleucine; #D2141-5 MG, Sigma), 10 μM 3-BP (3-Bromo-2-oxopropionic acid, #376817-M, Sigma), 10 mM Glutamate (#6106 Apr. 3, Sigma) before addition of targets.

siRNA experiments. Dendritic cells were treated with SMARTpool: Accell Slc7a11 siRNA (#E-047420-00-0010), Accell Slc7a1 siRNA (#E-042922-00-0005), Accell Slc7a5 siRNA (#E-041166-00-0010) or Accell Non-targeting siRNA #1 (#D-001910 Jan. 5) and Accell Non-targeting siRNA #4 (#D-001910-04-5) from Dharmacon, according to the manufacturer's instructions, 2 days before the engulfment assay.

qRT-PCR. Total RNA was extracted from cells using the RNeasy Mini Kit (Qiagen) and cDNA was synthesized using Sensifast cDNA Synthesis Kit (#BIO-650504), according to the manufacturer's instructions. Quantitative gene expression analysis for mouse genes was performed using mouse-sequence specific Taqman probes that are non-cross reactive with human sequences (Applied Biosystems), run on a Roche Lightcycler 480-384.

ELISA. GDF15 levels were measured in supernatants of dendritic cells or total skin lysates by using Mouse/Rat GDF-15 Quantikine ELISA Kit (#MGD150, R&D Systems). For skin lysates, total (shaved skin) was lysed in 50 mM Tris-HCl (pH 8,5) using the Precellys 24 tissue homogenizer and 25 μg lysate per sample were used for ELISA.

RNA sequencing. Wild type or Slc7a11-deficient BMDC were co-cultured with apoptotic Jurkat cells for 4 h, unbound Jurkat cells were removed by washing with PBS and engulfing BMDC were isolated by sorting with BD FACSAria™III Cell Sorter. Total RNA was extracted, and an mRNA library was prepared using the IlluminaNovoseq6000platform by Novogene. HISAT2 was selected to map the filtered sequenced reads to the reference genome. BAM files containing mapping results were counted using the feature Counts function in the R package Rsubread. Counting was performed using both mouse and human genomes for comparison although downstream analyses were only performed on mouse data. DEG analysis was then performed using DESeq2 considering all genes with FDR≤0.05 and 0.58≤Log2FC≤−0.58. All genes that resulted from the analysis were curated using multiple methods, including literature mining and function determination (known or predicted) via UniProt.

scRNA sequencing and analysis. Single-cell RNA-sequencing (scRNAseq) datasets on live cells sorted from control wild-type (WT: Cre-negative OTULIN^{fl/fl; n=}1) skin and lesional (L; n=3) and non-lesional (NL; n=2) Δ^{ker}OTULIN skin are fully described in Hoste et al., (Nature Communications, 2021, In Press).

Bioinformatics analysis. Gene sets were tested for unidirectional enrichment and visualized using the Triwise package. Gene expression data sets analyzed in RStudio Version 1.2.1335 and based on mean values of the normalized gene counts of each of three dendritic cell (DC) populations (lung, skin and spleen) extracted from ImmGen Microarray V1 data set “GSE15907”.

Mice. The following mouse lines were used: C57BL/6J, Slc7a11KO⁶³ and littermate wild-type, B6. BKS (D)-Leprdb/J (db/db) and PDGFRα-H2B-eGFP reporter mice⁶⁵. For the GDF15KO mice, the ES cells (as in ref⁶⁴) were obtained from EUCOMM and newly generated in our transgenic mouse core facility. Mice were housed in individually-ventilated cages at the VIB Center for Inflammation Research, in a specific pathogen-free animal facility. All experiments on mice were conducted according to institutional, national and European animal regulations. Animal protocols were approved by the ethics committee of Ghent University.

Skin wounding and erastin regimen. Full-thickness wounds were made as previously described^{65.56} Briefly, wounds were made on shaved back skin by using 8-mm punch biopsy needles (Stiefel Instruments) under analgesia and general anesthesia in 8-week-old female C57BL/6J, B6.BKS (D)-Leprdb/J (db/db), GDF15KO, Slc7a11KO and control littermates. For wound healing experiments, assuming standard deviations of 10-15%, an experimental group of n=10 is needed to obtain statistical power of 90% (significance level 0.05) and detect a difference between means of 20%, using two-tailed paired T-testing. No animals were excluded from any experiment.

For topical applications, mice were intradermally injected with Imidazole ketone erastin (IKE at 20 mg kg⁻¹ in 100 μl PBS; MedChemExpress, #HY-114481) or recombinant human GDF-15 (0.7 mg kg⁻¹ in 100 μl PBS; R&D Systems, #9279-GD-050) or RSL3 (20 mg kg-1 in 100 μl PBS; Sellechem, #S8155) or the respective vehicle controls (DMSO or 4 mM HCl in 100 μl PBS) at the time of wounding and for two consecutive days. For the experiments indicated as ‘erastin regimen’ or vehicle regimen, 5 million apoptotic Jurkat cells were administrated once on day 0 in 50 μl PBS on top of back skin immediately after full-thickness excision biopsy. For the mice injected intradermally with supernatants from efferocytic dendritic cells, supernatants from dendritic cells cultured with apoptotic Jurkat cells for 16 h were collected, centrifuged to eliminate debris and frozen or lyophilized for preservation.

Histology and Immunohistochemistry. Skin biopsies were fixed using 4% paraformaldehyde overnight. Following dehydration steps, samples were embedded in paraffin and sectioned at 10 μm thickness. Dewaxed paraffin skin sections were stained with Hematoxylin and Eosin stains or subjected to heat-mediated antigen retrieval (citrate buffer; pH=6), and apoptotic cells were evaluated with cleaved caspase-3 antibody (1:100; Cell Signaling Technology #9664). Slides were incubated with secondary antibody followed and peroxidase activity was detected with diaminobenzidine (DAB)-substrate kit (Cell signaling, #8059P). Nuclei were counterstained with Hematoxylin staining. Cleaved caspase-3⁺ positive cells were manually counted by using Zen software by Zeiss. Quantification of cleaved caspase-3⁺ was cells was done by an independent researcher, who was blinded to the genotypes or treatment.

Immunofluorescence. Dewaxed paraffin skin sections were subjected to heat-mediated antigen retrieval (citrate buffer; pH=6), blocked with 0.5% fish skin gelatin, 4% BSA in PBS and labelled with biotin anti-CD11cAb (1:200, BD Pharmingen #553800), anti-Slc7a11 Ab (1:200, in-house developed⁶⁷) or cleaved caspase-3 antibody (1:100; Cell Signaling Technology #9664). As secondary antibodies streptavidin 594 Alexafluor (1:2000) and donkey-anti-rabbit DyLight 488 (1:2000) were used in combination with DAPI.

Statistical analysis. Statistical significance was determined using GraphPad Prism 9, using unpaired Student's two-tailed t-test, one-way ANOVA or two-way ANOVA. * P<0.05, ** P<0.01, *** P<0.001, **** P<0.0001 were considered significant.

### REFERENCES

1 Henson, P. M. Cell Removal: Efferocytosis. Annu Rev Cell Dev Biol 33, 127-144, doi: 10.1146/annurev-cellbio-111315-125315 (2017).
2 Boada-Romero, E., Martinez, J., Heckmann, B. L. & Green, D. R. The clearance of dead cells by efferocytosis. Nat Rev Mol Cell Biol 21, 398-414, doi: 10.1038/s41580-020-0232-1 (2020).
3 Hughes, L. D., Wang, Y., Meli, A. P., Rothlin, C. V. & Ghosh, S. Decoding Cell Death: From a Veritable Library of Babel to Vade Mecum? Annu Rev Immunol 39, 791-817, doi: 10.1146/annurev-immunol-102819-072601 (2021).
4 Morioka, S., Maueroder, C. & Ravichandran, K. S. Living on the Edge: Efferocytosis at the Interface of Homeostasis and Pathology. Immunity 50, 1149-1162, doi: 10.1016/j.immuni.2019.04.018 (2019).
5 Doran, A. C., Yurdagul, A., Jr. & Tabas, I. Efferocytosis in health and disease. Nat Rev Immunol 20, 254-267, doi: 10.1038/s41577-019-0240-6 (2020).
6 Henson, P. M. & Hume, D. A. Apoptotic cell removal in development and tissue homeostasis. Trends Immuno/27, 244-250, doi: 10.1016/j.it.2006.03.005 (2006).
7 Cabeza-Cabrerizo, M., Cardoso, A., Minutti, C. M., Pereira da Costa, M. & Reis, E. S. C. Dendritic Cells Revisited. Annu Rev Immunol 39, 131-166, doi: 10.1146/annurev-immunol-061020-053707(2021).
8 Guermonprez, P. & Amigorena, S. Pathways for antigen cross presentation. Springer Semin Immunopathol 26, 257-271, doi: 10.1007/s00281-004-0176-0 (2005).
9 Albert, M. L., Sauter, B. & Bhardwaj, N. Dendritic cells acquire antigen from apoptotic cells and induce class I-restricted CTLs. Nature 392, 86-89, doi: 10.1038/32183 (1998).
10 Inaba, K. et al. Efficient presentation of phagocytosed cellular fragments on the major histocompatibility complex class II products of dendritic cells. J Exp Med 188, 2163-2173, doi: 10.1084/jem.188.11.2163 (1998).
11 Gallucci, S., Lolkema, M. & Matzinger, P. Natural adjuvants: endogenous activators of dendritic cells. Nat Med 5, 1249-1255, doi: 10.1038/15200 (1999).
12 Blander, J. M. & Medzhitov, R. On regulation of phagosome maturation and antigen presentation. Nat Immunol 7, 1029-1035, doi: 10.1038/ni1006-1029 (2006).
13 Khanna, S. et al. Macrophage dysfunction impairs resolution of inflammation in the wounds of diabetic mice. PLOS One 5, e9539, doi: 10.1371/journal.pone.0009539 (2010).
14 Wetzler, C., Kampfer, H., Stallmeyer, B., Pfeilschifter, J. & Frank, S. Large and sustained induction of chemokines during impaired wound healing in the genetically diabetic mouse: prolonged persistence of neutrophils and macrophages during the late phase of repair. J Invest Dermatol 115, 245-253, doi: 10.1046/j.1523-1747.2000.00029.x (2000).
15 Moulik, P. K., Mtonga, R. & Gill, G. V. Amputation and mortality in new-onset diabetic foot ulcers stratified by etiology. Diabetes Care 26, 491-494, doi: 10.2337/diacare.26.2.491 (2003).
16 Lenz, A., Heine, M., Schuler, G. & Romani, N. Human and murine dermis contain dendritic cells. Isolation by means of a novel method and phenotypical and functional characterization. J Clin Invest 92, 2587-2596, doi: 10.1172/JCI116873 (1993).
17 Seneschal, J., Clark, R. A., Gehad, A., Baecher-Allan, C. M. & Kupper, T. S. Human epidermal Langerhans cells maintain immune homeostasis in skin by activating skin resident regulatory T cells. Immunity 36, 873-884, doi: 10.1016/j.immuni.2012.03.018 (2012).
18 Mirza, R., DiPietro, L. A. & Koh, T. J. Selective and specific macrophage ablation is detrimental to wound healing in mice. Am J Pathol 175, 2454-2462, doi: 10.2353/ajpath.2009.090248 (2009).
19 Shook, B., Xiao, E., Kumamoto, Y., Iwasaki, A. & Horsley, V. CD301b+Macrophages Are Essential for Effective Skin Wound Healing. J Invest Dermatol 136, 1885-1891, doi: 10.1016/j.jid.2016.05.107 (2016).
20 Phillipson, M. & Kubes, P. The Healing Power of Neutrophils. Trends Immunol 40, 635-647, doi: 10.1016/j.it.2019.05.001 (2019).
21 Morioka, S. et al. Efferocytosis induces a novel SLC program to promote glucose uptake and lactate release. Nature 563, 714-718, doi: 10.1038/s41586-018-0735-5 (2018).
22 Kelly, B. & Pearce, E. L. Amino Assets: How Amino Acids Support Immunity. Cell Metob 32, 154-175, doi: 10.1016/j.cmet.2020.06.010 (2020).
23 Procaccini, C. et al. Signals of pseudo-starvation unveil the amino acid transporter SLC7A11 as key determinant in the control of Treg cell proliferative potential. Immunity, doi: 10.1016/j.immuni.2021.04.014 (2021).
24 D′Angelo, J. A. et al. The cystine/glutamate antiporter regulates dendritic cell differentiation and antigen presentation. J Immunol 185, 3217-3226, doi: 10.4049/jimmunol.1001199 (2010).
25 Fotiadis, D., Kanai, Y. & Palacin, M. The SLC3 and SLC7 families of amino acid transporters. Mol Aspects Med 34, 139-158, doi: 10.1016/j.mam.2012.10.007 (2013).
26 Merckx, E. et al. Absence of system xc (-) on immune cells invading the central nervous system alleviates experimental autoimmune encephalitis. J Neuroinflammation 14, 9, doi: 10.1186/s12974-016-0787-0 (2017).
27 Massie, A. et al. Time-dependent changes in striatal xCT protein expression in hemi-Parkinson rats. Neuroreport 19, 1589-1592, doi: 10.1097/WNR.0b013e328312181c (2008).
28 Mesci, P. et al. System xC-is a mediator of microglial function and its deletion slows symptoms in amyotrophic lateral sclerosis mice. Brain 138, 53-68, doi: 10.1093/brain/awu312 (2015).
29 Lin, C. H. et al. Decreased mRNA expression for the two subunits of system xc(-), SLC3A2 and SLC7A11, in WBC in patients with schizophrenia: Evidence in support of the hypo-glutamatergic hypothesis of schizophrenia. J Psychiatr Res 72, 58-63, doi: 10.1016/j.jpsychires.2015.10.007(2016).
30 Massie, A. et al. Dopaminergic neurons of system x(c)(-)-deficient mice are highly protected against 6-hydroxydopamine-induced toxicity. FASEB J 25, 1359-1369, doi: 10.1096/fj.10-177212(2011).
31 Kaleeba, J. A. & Berger, E. A. Kaposi's sarcoma-associated herpesvirus fusion-entry receptor: cystine transporter xCT. Science 311, 1921-1924, doi: 10.1126/science.1120878 (2006).
32 Kandasamy, R. K. et al. A time-resolved molecular map of the macrophage response to VSV infection. NPJ Syst Biol Appl 2, 16027, doi: 10.1038/npjsba.2016.27 (2016).
33 Rabinowitz, J. et al. xCT/SLC7A11 antiporter function inhibits HIV-1 infection. Virology 556, 149-160, doi: 10.1016/j.virol.2021.01.008 (2021).
34 Robert, S. M. et al. SLC7A11 expression is associated with seizures and predicts poor survival in patients with malignant glioma. Sci Transl Med 7, 289ra286, doi: 10.1126/scitranslmed.aaa8103 (2015).
35 Koppula, P., Zhuang, L. & Gan, B. Cystine transporter SLC7A11/xCT in cancer: ferroptosis, nutrient dependency, and cancer therapy. Protein Cell, doi: 10.1007/s13238-020-00789-5 (2020).
36 Lin, W. et al. SLC7A11/xCT in cancer: biological functions and therapeutic implications. Am J Cancer Res 10, 3106-3126 (2020).
37 Zhao, Y. et al. The Role of Erastin in Ferroptosis and Its Prospects in Cancer Therapy. Onco Targets Ther 13, 5429-5441, doi: 10.2147/OTT.S254995 (2020).
38 Hassannia, B., Vandenabeele, P. & Vanden Berghe, T. Targeting Ferroptosis to Iron Out Cancer. Cancer Cell 35, 830-849, doi: 10.1016/j.ccell.2019.04.002 (2019).
39 Conrad, M. & Pratt, D. A. The chemical basis of ferroptosis. Not Chem Biol 15, 1137-1147, doi: 10.1038/s41589-019-0408-1 (2019).
40 Jiang, X., Stockwell, B. R. & Conrad, M. Ferroptosis: mechanisms, biology and role in disease. Nat Rev Moi Cell Biol 22, 266-282, doi: 10.1038/s41580-020-00324-8 (2021).
41 Szondy, Z., Garabuczi, E., Joos, G., Tsay, G. J. & Sarang, Z. Impaired clearance of apoptotic cells in chronic inflammatory diseases: therapeutic implications. Front Immunol 5, 354, doi: 10.3389/fimmu.2014.00354 (2014).
42 Rajesh, A. et al. Depletion of langerin (+) cells enhances cutaneous wound healing. Immunology 160, 366-381, doi: 10.1111/imm.13202 (2020).
43 Rajesh, A. et al. Skin antigen-presenting cells and wound healing: New knowledge gained and challenges encountered using mouse depletion models. Immunology 163, 98-104, doi: 10.1111/imm.13311 (2021).
44 Lachmann, A. et al. Massive mining of publicly available RNA-seq data from human and mouse. Nat Commun 9, 1366, doi: 10.1038/s41467-018-03751-6 (2018).
45 Larraufie, M. H. et al. Incorporation of metabolically stable ketones into a small molecule probe to increase potency and water solubility. Bioorg Med Chem Lett 25, 4787-4792, doi: 10.1016/j.bmcl.2015.07.018 (2015).
46 Zhang, Y. et al. Imidazole Ketone Erastin Induces Ferroptosis and Slows Tumor Growth in a Mouse Lymphoma Model. Cell Chem Biol 26, 623-633 e629, doi: 10.1016/j.chembiol.2019.01.008(2019).
47 Yang, W. S. et al. Regulation of ferroptotic cancer cell death by GPX4. Cell 156, 317-331, doi: 10.1016/j.cell.2013.12.010 (2014).
48 Keramati, A. R. et al. A form of the metabolic syndrome associated with mutations in DYRK18. N Engl J Med 370, 1909-1919, doi: 10.1056/NEJMoa1301824 (2014).
49 Honma, K., Kamikubo, M., Mochizuki, K. & Goda, T. Insulin-induced inhibition of gluconeogenesis genes, including glutamic pyruvic transaminase 2, is associated with reduced histone acetylation in a human liver cell line. Metabolism 71, 118-124, doi: 10.1016/j.metabol.2017.03.009 (2017).
50 Nakayasu, E. S. et al. Comprehensive Proteomics Analysis of Stressed Human Islets Identifies GDF15 as a Target for Type 1 Diabetes Intervention. Cell Metab 31, 363-374 e366, doi: 10.1016/j.cmet.2019.12.005 (2020).
51 He, X. et al. The association between serum growth differentiation factor 15 levels and lower extremity atherosclerotic disease is independent of body mass index in type 2 diabetes. Cardiovasc Diabetol 19, 40, doi: 10.1186/s12933-020-01020-9 (2020).
52 Beale, E. G., Harvey, B. J. & Forest, C. PCK1 and PCK2 as candidate diabetes and obesity genes. Cell Biochem Biophys 48, 89-95, doi: 10.1007/s12013-007-0025-6 (2007).
53 Thwe, P. M. et al. Cell-Intrinsic Glycogen Metabolism Supports Early Glycolytic Reprogramming Required for Dendritic Cell Immune Responses. Cell Metab 26, 558-567 e555, doi: 10.1016/j.cmet.2017.08.012 (2017).
54 Han, G. & Ceilley, R. Chronic Wound Healing: A Review of Current Management and Treatments. Adv Ther 34, 599-610, doi: 10.1007/s12325-017-0478-y (2017).
55 Zhao, G. et al. Delayed wound healing in diabetic (db/db) mice with Pseudomonas aeruginosa biofilm challenge: a model for the study of chronic wounds. Wound Repair Regen 18, 467-477, doi: 10.1111/j.1524-475X.2010.00608.x (2010).
56 Bonnefoy, F. et al. Factors Produced by Macrophages Eliminating Apoptotic Cells Demonstrate Pro-Resolutive Properties and Terminate Ongoing Inflammation. Front Immunol 9, 2586, doi: 10.3389/fimmu.2018.02586 (2018).
57 Pakyari, M., Farrokhi, A., Maharlooei, M. K. & Ghahary, A. Critical Role of Transforming Growth Factor Beta in Different Phases of Wound Healing. Adv Wound Care (New Rochelle) 2, 215-224, doi: 10.1089/wound.2012.0406 (2013).
58 Emmerson, P. J., Duffin, K. L., Chintharlapalli, S. & Wu, X. GDF15 and Growth Control. Front Physiol 9, 1712, doi: 10.3389/fphys.2018.01712 (2018).
59 Coll, A. P. et al. GDF15 mediates the effects of metformin on body weight and energy balance. Nature 578, 444-448, doi: 10.1038/s41586-019-1911-y (2020).
60 Deckers, J. et al. Co-Activation of Glucocorticoid Receptor and Peroxisome Proliferator-Activated Receptor-gamma in Murine Skin Prevents Worsening of Atopic March. J Invest Dermatol 138, 1360-1370, doi: 10.1016/j.jid.2017.12.023 (2018).
61 Sepulveda, F. E. et al. Critical role for asparagine endopeptidase in endocytic Toll-like receptor signaling in dendritic cells. Immunity 31, 737-748, doi: 10.1016/j.immuni.2009.09.013 (2009).
62 Wiernicki, B. et al. Excessive phospholipid peroxidation distinguishes ferroptosis from other cell death modes including pyroptosis. Cell Death Dis 11, 922, doi: 10.1038/s41419-020-03118-0 (2020).
63 Sato, H. et al. Redox imbalance in cystine/glutamate transporter-deficient mice. J Biol Chem 280, 37423-37429, doi: 10.1074/jbc.M506439200 (2005).
64 Lambrecht, S. et al. Growth differentiation factor 15, a marker of lung involvement in systemic sclerosis, is involved in fibrosis development but is not indispensable for fibrosis development. Arthritis Rheumatol 66, 418-427, doi: 10.1002/art.38241 (2014).
65 Van Hove, L. et al. Fibrotic enzymes modulate wound-induced skin tumorigenesis. EMBO Rep 22, e51573, doi: 10.15252/embr.202051573 (2021).
66 Hoste, E. et al. Innate sensing of microbial products promotes wound-induced skin cancer. Nat Commun 6, 5932, doi: 10.1038/ncomms6932 (2015).
67 Van Liefferinge, J. et al. Comparative analysis of antibodies to xCT (Slc7a11): Forewarned is forearmed. J Comp Neurol 524, 1015-1032, doi: 10.1002/cne.23889 (2016).

1. A composition comprising an inhibitor of Slc7a11 and apoptotic cells.

2. The composition of claim 1, wherein the composition is formulated as a medicament.

3. A method of promoting wound healing in a mammal, the method comprising: administering to the mammal the composition of claim 1.

4. The method according to claim 3, wherein the wound is a chronic wound.

5. The composition of claim 1, wherein the inhibitor of Slc7a11 is selected from a small compound, an antibody, a gapmer, a shRNA, a synthetic siRNA, an anti-sense oligonucleotide selected from a locked nucleic acid (LNA), a peptide nucleic acid (PNA) or a morpholino, a CRISPR, a TALEN, or a Zinc-finger nuclease targeting Slc7a11.

6. The method according to claim 4, wherein chronic wound is diabetic wound, an arterial or venous wound, or a pressure wound.

7. composition of claim 5, wherein the composition further comprises GDF-15.

8. The method according to claim 3, wherein the composition further comprises GDF-15.

9. The method according to claim 8 wherein the wound is a chronic wound.

10. The method according to claim 8, wherein the inhibitor of slc7a11 is selected from a small compound, an antibody, a gapmer, a shRNA, a synthetic siRNA, an anti-sense oligonucleotide selected from a locked nucleic acid (LNA), a peptide nucleic acid (PNA) or a morpholino, a CRISPR, a TALEN, or a Zinc-finger nuclease targeting Slc7a11.

11. The method according to claim 9, wherein the chronic wound is a diabetic wound, an arterial or venous wound, or a pressure wound.

12. (canceled)

13. (canceled)

14. The method according to claim 3, wherein the inhibitor of slc7a11 is selected from a small compound, an antibody, a gapmer, a shRNA, a synthetic siRNA, an anti-sense oligonucleotide selected from a locked nucleic acid (LNA), a peptide nucleic acid (PNA) or a morpholino, a CRISPR, a TALEN, or a Zinc-finger nuclease targeting Slc7a11.
